(19) United States
(12) Patent Application Publication
(10) Pub. No.: US 2003/0017467 A1
(43) Pub. Date: Jan. 23, 2003

(54) Multiple-site sample-handling apparatus and method

(75) Inventors: Herbert H. Hooper, Belmont, CA (US); Sharat Singh, San Jose, CA (US); Travis D. Boone, San Mateo, CA (US); Rolfe Anderson, Saratoga, CA (US); David Albagli, Millbrae, CA (US); Antonio J. Ricco, Los Gatos, CA (US)

Correspondence Address: PERKINS COIE LLP, P.O. BOX 2168, MENLO PARK, CA, 94026, US

(73) Assignee: Aclara BioSciences, Inc.

(21) Appl. No.: 09/938,439

(22) Filed: Aug. 23, 2001

Related U.S. Application Data
(63) Continuation-in-part of application No. 09/788,209, filed on Feb. 16, 2001.
(60) Provisional application No. 60/183,626, filed on Feb. 18, 2000.

(51) Int. Cl. G01N 33/00

(52) U.S. Cl. 435/6; 436/180; 422/58; 422/100

(57) ABSTRACT

A microchannel apparatus and method for processing a sample are disclosed. The apparatus include a multisite reaction channel, one or more sample-preparation stations upstream of the reaction channel, for carrying out one or more selected sample-preparation steps effective to convert a sample to the bulk-phase medium, and one or more product-processing stations downstream of the reaction channel, for processing products generated in one or more of the reaction regions. Also included is structure for transferring solvent or solvent components between one of the sample-preparation stations and one or more selected reaction regions in the reaction channel, and between one or more selected reaction regions in the reaction channel and one of said product-processing stations, under the control of a control unit.

This application is a continuation of U.S. patent application for Multiple-Site Reaction Device and Method, Ser. No. 09/788,209, filed Feb. 16, 2001, which is incorporated herein by reference in its entirety.

### FIELD OF THE INVENTION

The present invention relates to small-volume reaction devices, and in particular to a device having a multiple-site reaction chamber in which a plurality of small-volume reactions can be carried out simultaneously, and to methods employing the device.

### BACKGROUND OF THE INVENTION

The conjunction of increasing biological targets and compounds for potentially modulating the activity of the targets requires new ways to perform assays. The recognition of single nucleotide polymorphisms (“snps”) as a potential source to screen genomes for traits related to responses to drugs, susceptibility to disease, physical capacity, and the like, creates a need for methodologies to determine the snps. The increasing interest in elucidating the numerous biological pathways in plants, animals and single celled species requires improvements in the performance of numerous determinations associated with molecular interactions, such as protein-protein binding, ligand-protein binding and protein-nucleic acid binding.

As the number of operations increases, there are many reasons for wanting to be able to carry out determinations in small volumes. Small volumes offer many advantages, not the least of which are reduced amount of reagents, speed for the reactions to occur, increased number of determinations within a small area, and the reduced size of equipment in relation to the number of determinations performed. The amount of reagent is important, since many of the protein targets are only difficult and costly to produce. For candidate compounds, frequently drugs, which are increasingly coming from combinatorial libraries, the amounts available for the first screen are extremely small. With the large number of compounds produced from a combinatorial library, it is of interest to be able to run as many as possible simultaneously or at least consecutively within a short period of time. The large number of proteins present in a cell and the nature of their interactions with other naturally occurring or synthetic compounds offers a major challenge in being able to screen individual proteins against a large library of other compounds.

Toward the end of reducing volumes in which determinations are carried out, a number of investigators have reported the use of capillary electrokinesis on a small substrate, where the channels and reservoirs are of sub millimeter dimensions. These approaches tend to involve individual operations for each unit, even though there may be common reagents. In addition, the necessity for a voltage source can have a negative effect on the determination. Illustrative approaches may be found in U.S. Pat. Nos. 5,876,946; 5,872,010; and 5,922,604; and PCT applications nos. WO99/51772; 99/34920; 99/09042; 99/11373; 98/52691; and 98/00231.There is, therefore, substantial interest in developing new techniques that provide for mesoscale operations in an efficient and economical manner.

There is substantial interest in being able to perform multiple reactions in nanoliter-scale volumes simultaneously, where each of the reactions may be addressed individually. Such systems would provide for reagent savings, increased sensitivity, direct comparisons, and the like. Operations should include the polymerase chain reaction, binding, enzyme reactions, identification of nucleic acid sequences or single nucleotide polymorphisms, etc.

PCT WO99/34920 describes a platen having a plurality of through-holes as a holder for individual reaction volumes of less than 100 nl. U.S. Pat. Nos. 5,837,551; 5,834,319; 5,807,755; 5,599,720; 5,516,635; 5,4432,099; 5,304,498; and 4,745,072 are a series of patents by Roger P. Ekins of assays employing spatially separated locations. See also, U.S. Pat. No. 4,491,570. PCT/WO/98/49344 describes a method for analyzing nucleic acids with a plurality of nucleic probes as specific sites in a channel.

### SUMMARY OF THE INVENTION

The invention includes, in one aspect, a microchannel apparatus for processing a sample. The apparatus include a microchannel device having a substrate, and formed in the substrate, an elongate or planar multisite reaction channel for receiving a bulk-phase medium containing sample components. The reaction channel has a plurality of reaction regions and region-specific reagents associated with each region, for simultaneously conducted different reactions on sample components within the reaction channel. Also included in the device are one or more sample-preparation stations, upstream of the reaction channel, for carrying out one or more selected sample-preparation steps effective to convert a sample to the bulk-phase medium, and one or more product-processing stations downstream of the reaction channel, for processing products generated in one or more of the reaction regions.

The apparatus further includes structure for transferring solvent or solvent components between one of the sample-preparation stations and one or more selected reaction regions in the reaction channel, and between one or more selected reaction regions in the reaction channel and one of said product-processing stations. A control unit in the apparatus is designed to activate the transfer structure, to effect transfer, in a selected reaction region, of solvent or solvent components from or to each hold or region-specific reservoir, to or from the associated reaction region.

The microchannel device in the apparatus may include a second reaction channel for receiving a second bulk-phase medium containing sample components, where this second channel has a plurality of reaction regions and region-specific reagents associated with each region, for simultaneously conducting different reactions on sample components within the reaction channel. One or more of the reaction regions in the first reaction channel may be operatively connected to associated reaction regions in the second reaction channel via gated side channels. The control unit in this embodiment is operative to transfer reaction components directly between associated reaction regions in the two reaction channels.

Alternatively, the reaction regions in the first-mentioned reaction channel may be operatively connected to reaction regions in the second reaction channel via a common hold reservoir which receives sample components from reaction regions in one reaction channel, and supplies the combined components to reaction regions in the other reaction channel.

In either embodiment, the reaction chambers may be formed in different layers of the device, with reaction regions in the two channels being interconnected by side channels extending between the two layers. At least one of the two reaction channels may include capillary-tube ports adapted to receive a capillary tube therein, for supplying or removing a selected reagent or component to or from that port.

At least some of the stations and the reaction channel may include capillary-tube ports adapted to receive a capillary tube therein, for supplying or removing a selected reagent or component to or from that port.

The sample-preparation stations include at least one of a cell-culture station, a station at which cells grown in the cell-culture station are lysed, and a reservoir containing lysing medium.

The product-processing stations include at least one of a (a) a waste reservoir for receiving selected components from the reaction regions; (b) a capture station at which selected components from the reaction channel are captured and concentrated; (c) an assay-reagent reservoir from which assay reagents are added to sample components; (d) an assay station from which assay components are added to sample components; (e) a separation channel at which sample components can be separated; and (f) a second multisite reaction channel.

The apparatus may further includes a detector for detecting the presence of absence of selected components contained in selected reaction regions. The control unit in this embodiment is operable to move selected reaction components to other stations in the device, based on the presence of absence of such detected components.

Also disclosed is a method of analyzing components in a sample. The method includes applying the sample to an inlet port in the above apparatus, operating the apparatus to process the sample, forming a bulk-phase medium containing sample components, operating the apparatus to transfer the bulk phase medium into the multisite reaction channel in the apparatus, under conditions that promote simultaneous reactions with sample components and region-specific reagents in the channel, and operating the apparatus to transfer one or more reacted components from the reaction channel into a processing station, to achieve at least one of the following processing results: (a) removal of one or more reacted components produced in the reaction channel; (b) assay of one or more reacted components produced in the reaction channel; (c) further reaction of one or more reacted components produced in the reaction channel, (d) mixing of two or more selected components contained or produced in the reaction channel; (e) transfer of one or more selected components produced in the reaction channel to selected reaction region(s) in a second multisite reaction channel.in the device; (f) separation of components contained in the reaction channel, or produced subsequently in the device, a separation medium; and (g) detection of components contained in the reaction channel, or produced subsequently in the device, by a detector in the device.

The method may include assaying one or more components produced in selected regions of the reaction channel; and based on the results of the assaying, transferring selected components to another station in the device.

These and other objects and features of the invention will become more fully apparent when the following detailed description of the invention is read in conjunction with the accompanying drawings.

### BRIEF DESCRIPTION OF THE DRAWINGS

FIGS. 1A, 1B, and 1C are plan (1A and 1B) and sectional (1C) views of a multisite microfluidics device constructed in accordance with one general embodiment of the present invention;

FIG. 1C FIGS. 2A and 2B are plan and sectional views of a multisite microfluidics device constructed in accordance with another general embodiment of the invention;

FIG. 3 shows a portion of a card with a plurality of reaction devices formed therein;

FIGS. 4A and 4B show steps in introducing fluid into one of the channels in the FIG. 1A device;

FIGS. 5A-5D illustrate exemplary methods for removing liquid from a channel in the FIG. 1A device;

FIGS. 6A-6D show steps in introducing fluid into and removing fluid from one of the channels in another embodiment of a card device in accordance with the invention;

FIGS. 7A-7C show alternative methods for releasably binding reaction-specific reagents, e.g., nucleic acids to the wall portion of a reaction region in the device of the invention;

FIG. 8 shows three adjacent wall portions in a channel, in accordance with the invention, illustrating three different-sequence nucleic acid primers releasably immobilized to the reaction-site wall portions through site-specific nucleic acids immobilized on the wall portions of the three sites;

FIGS. 9A-9E illustrate steps in carrying out simultaneous PCR reactions in accordance with the invention; and

FIGS. 10A-10C illustrate steps in carrying out simultaneous PCR reactions in accordance with the invention;

FIGS. 11A-11C illustrate a portion of a reaction channel in a multisite reaction device, showing three reaction regions therein, and associated reservoirs on either sides of each channel, and illustrating the condition of the device during a multiplexed reaction (11A), when transferring one reaction product out of a selected region in the reaction channel (11B), and when adding a region-specific reagent to each region (11C);

FIGS. 12A and 12B are cross-sectional views taken along line 12-12 in FIG. 11A, in a device having either self-contained reservoirs and electrodes for moving material from one reservoir into a channel reaction region, or from a channel region into one reservoir, either electrokinetically (12A) or by capillary or pressure forces (12B);

FIG. 13 illustrates a portion of a reaction channel in a multisite reaction device, showing three reaction regions therein, and associated reservoirs on either sides of each channel, where the pairs of reservoirs on one side of the channel are connected to a pressure or vacuum channel through controllable valves;

FIGS. 14A-14C illustrate various types of reaction or product manipulations possible in the device of FIGS. 11A-11C and FIG. 12;

FIGS. 15A-15C illustrate a portion of a reaction channel in a multisite reaction device, showing three reaction regions therein, and associated individual-region reservoirs on one side of the channel, and a common mixing reservoir on the other side of the channel, and illustrating the condition of the device during a multiplexed reaction (15A), when transferring one reaction product out of a selected region in the reaction channel by electrokinetic sample movement (15B) or pressure-differential sample movement (15C);

FIGS. 16A-16C illustrate various types of reaction or product manipulations possible in the device of FIGS. 15A-15C;

FIG. 17 illustrates a portion of a reaction channel in a multisite reaction device, showing three reaction regions therein;

FIGS. 18A and 18B illustrate various types of reaction or product manipulations possible in the device of FIG. 17;

FIG. 19 illustrates a portion of a reaction channel like the one shown in FIG. 17, but where the common mixing reservoirs on either side of the channel are controlled by individual valves;

FIG. 20 shows portions of two reactions channels in a device constructed according to another general embodiment of the invention, and reservoirs and side channel connections between corresponding reaction regions in the two channels;

FIG. 21 shows portions of two reactions channels in a device constructed according to a related embodiment of the invention, where the reaction regions in each channel are individually controlled for movement of material from a common supply reservoir to a common mixing reservoir, and the mixing reservoir of the first channel is the supply channel of the second;

FIGS. 22A-22C illustrate various types of reaction or product manipulations possible in the device of FIG. 21;

FIG. 23 illustrates a two-layer device having multisite reaction channels formed in upper and lower layers of the device, where the upper channel communicates with reservoirs that are removably insertable into openings in the upper layer, the lower channel communicates with reservoirs that are removably insertable into openings in the lower layer, and the two channels communicate with each other through internal connecting channels;

FIG. 24 illustrates a multi-function device containing a multisite reaction channel and upstream and downstream processing stations; and

FIG. 25 is a flow diagram of exemplary operations that can be carried out in the device of claim 24.

### DETAILED DESCRIPTION OF THE INVENTION

I. Definitions

Unless otherwise indicated, the terms below have the following definitions herein.

An “elongate channel” is a substantially one-dimensional channel having a length dimension that is at least 1-2 orders of magnitude greater than the width dimension of the channel. The channel may be linear or curved, e.g., spiral or serpentine. The channel has preferred width and depth dimensions between 20-1,000 microns, typically 25-500 microns, and a length of up several cm's or more. A channel having these depth and width dimensions is also referred to herein as an elongate microchannel.

A “planar channel” is a sheetlike channel formed between two closely spaced planar expanses, e.g., plates whose confronting surfaces are spaced 20-1,000 microns, typically 50-500 microns from one another. A channel having these between-plate spacings is also referred to herein as a planar microchannel.

A “bulk-phase reaction medium” is an aqueous solution containing one or more reagents that are common to different reactions carried out in the device of the invention. For example, for carrying out PCR reactions in the device, the bulk-phase medium will typically contain target DNA to be amplified, DNA polymerase, all four nucleotide triphosphates and other components needed, in combination with reagent(s) supplied in each reaction region, e.g., DNA primers, for carrying out the desired reaction.

A channel is “dimensioned to substantially prevent convective flow” if the spacing between confronting walls of the channel (either elongate or planar) are such as to limit the mixing of solute molecules within the channel to diffusional mixing, as opposed to convective mixing within the bulk phase. Channels having width and depth dimensions in the 20-1,000 micron, preferably 50-500 micron size range and planar channels having between-plate spacing in the same dimension ranges are so dimensioned.

“Small-volume reaction regions” refers to reaction regions having volumes of about 1 microliter or less, typically 25-600 nanoliter.

“Discrete reaction regions” means that at least some reaction regions are spaced one from another in a channel. Preferably, each reaction region is spaced apart from all other regions in the channel.

A “sequence-specific nucleic acid reaction” is one that occurs only when a target DNA reactant contains a specific sequence. Such reactions include, without limitation, primer-initiated polymerization or ligase reactions, polymerase chain reaction (PCR), primer-dependent 5′-exonuclease reactions, and restriction endonuclease reactions. “Region-specific nucleic acids” refers to oligonucleotide or polynucleotide molecules that have a selected sequence or region of sequence that is different for different reaction sites, thus allowing different sequence-dependent reactions to occur in the different reaction regions of the device of the invention.

“Releasably bound”, as applied to one or more reagents, means that the reagent(s) remain bound to the wall portion, when a bulk-phase medium is introduced into a reaction site, but are released into the bulk phase medium either passively over time, or actively by the application of heat, light or other external stimulus, or by the inclusion in the bulk phase of specific cleavage agents, such as a reducing agent or hydrolytic enzymes. As used herein, the term is synonymous with “releasably and non-diffusably bound”, where a reagent is non-diffusably bound if it is not released from a reaction-region wall portion upon initial hydration with bulk-phase medium.

A “microfluidics device” is a device having channels, preferably enclosed with width and depth dimensions in the 20-1,000 micron, preferably 50-500 micron size range and planar channels having between-plate spacing in the same dimension ranges.

II. Multisite Reaction Device

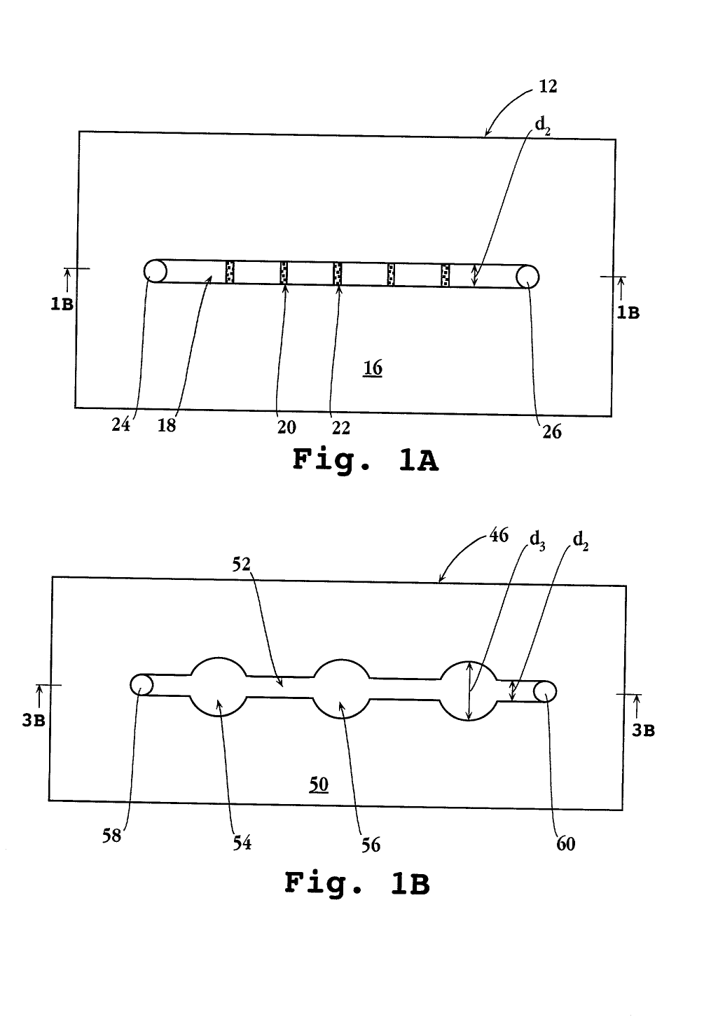

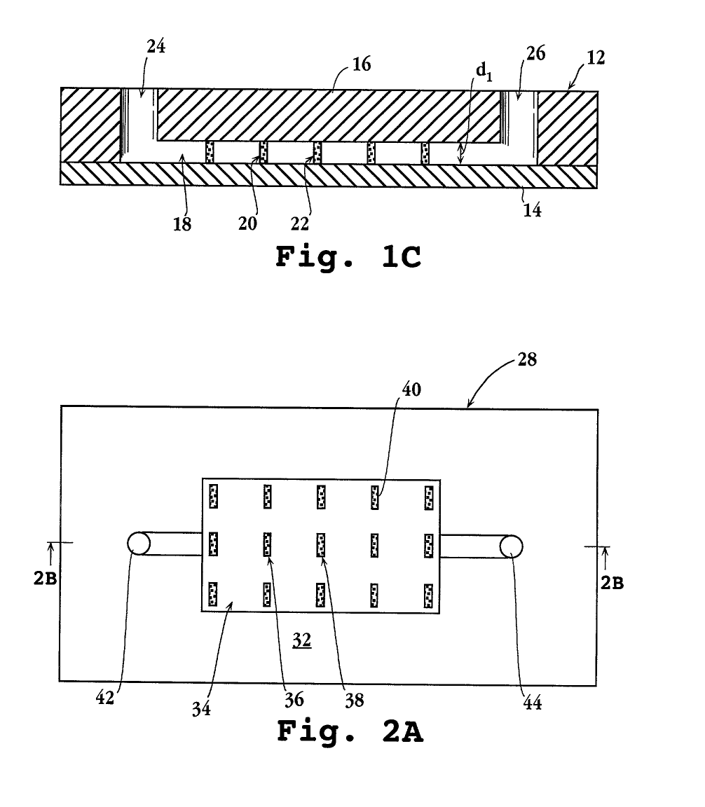

FIGS. 1A and 1CB are plan and sectional views, respectively, of a device 12 constructed according to an embodiment of the invention, for carrying out a plurality of different reactions in a single bulk-phase reaction medium. The device includes a substrate 14 and a covering 16 which is attached, as by thermal welding or the like to the substrate. Formed in the covering is a channel 18 extending between an input port 24 and an output port 26. As can be appreciated, the substrate serves to enclose the channel, confining liquid movement within the channel through ports 24, 26. Alternatively, the channel may be formed in the substrate and enclosed by the covering over the substrate. The substrate and covering thus provide means defining an elongate channel in the device. Other channel-defining means can include a tube, such as a capillary tube, an integral molded structure with an internal microchannel.

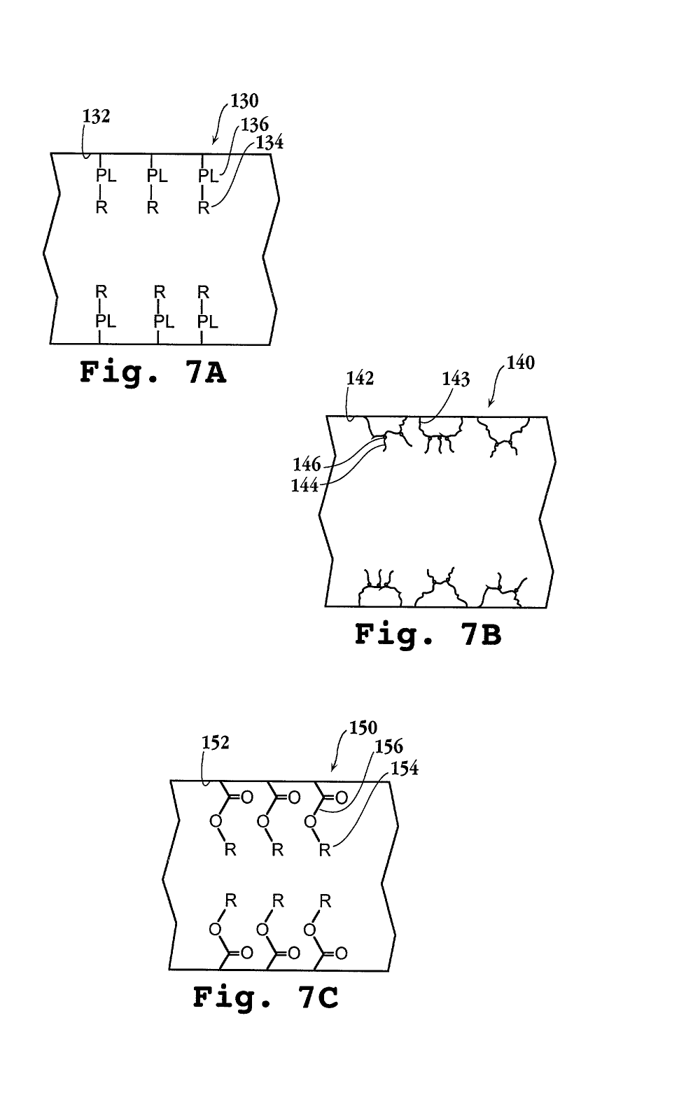

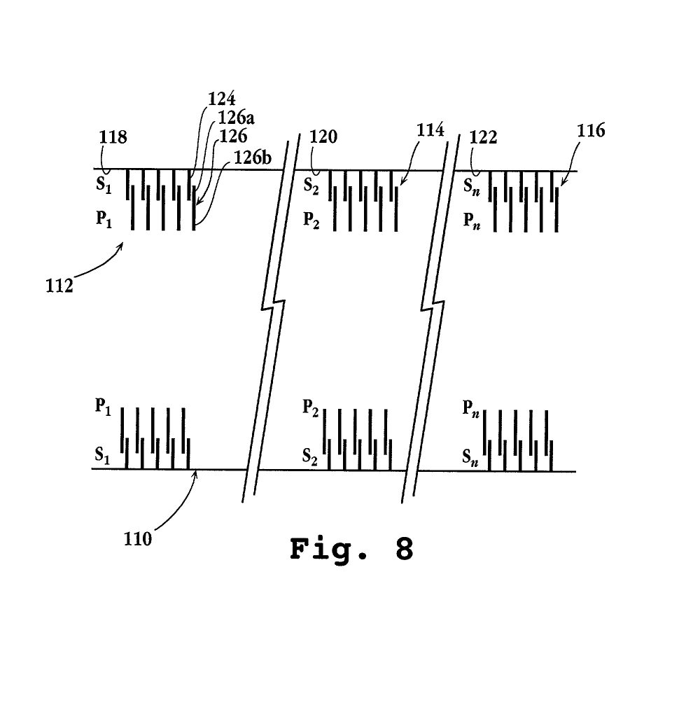

According to an important aspect of the invention, the device includes a plurality of discrete reactions regions, such as regions 20, 22, within the channel, at spaced positions along the length of the channel. The portion of the channel extending through the reaction regions has a wall portion, such as the top or side channel wall portions formed in covering 16, to which reaction-specific reagent(s) are releasably attached. As will be considered below with reference to FIGS. 7 and 8, the reagent(s) are released after bulk-phase medium is introduced into the channel, providing reactant(s) that are specific for each reaction site. The reagent(s) react in solution with reactants contained in the bulk-phase (and thus present at all reaction sites) in a reaction that is site specific, that is, determined by the reagent(s) released in each site.

The channel is dimensioned in width and depth to substantially prevent convective fluid flow between adjacent reaction sites. That is, to the extent reactants in each reaction site are able to mix over the course of the reaction carried out in each site, such mixing occurs primarily by diffusion of solute components rather than by bulk-phase stirring by convection. This feature limits the spread of solute reaction components, including reaction products, to that site and, at most, adjacent sites.

To this end, the channel is generally of a cross-sectional area of not more than about 1 mm², usually less than about 0.8 mm², preferably less than about 0.4 mm², and frequently as small as about 50μ²or in some situations, may even be less. The cross-section may be circular or non-circular. For non-circular cross-sections the channels will generally have an average depth of about 5μ to 1 mm, preferably in the range of about 5 to 500μ, more usually 100 to 300μ, and an average width in the range of about 10μ to 1 mm, more usually 25 to 500μ. Selection of the size of the channel will depend on the reaction volume desired, the nature of the signal to be detected, the sensitivity of the detection system, and the like.

The length of the channel will usually be at least about 0.5 cm, usually at least about 1 cm, and may be 20 cm or more, usually not more than about 10 cm. The length will be, to a degree, dependent on the number of reaction regions, the length of the individual regions, and the separation between regions. Although a linear channel is shown, it will be appreciated that other elongate channel configurations are possible, e.g., a serpentine or spiral channel, and these more compact channel shapes will generally be desirable when the device is constructed in microchip form, e.g., on a surface having an area of 1 cm² or less.

Desirably, the reaction volume of each reaction region will be in the range of about 5 nl to 900 nl, usually in the range of about 5 nl to 600 nl, more usually in the range of about 10 nl to 300 nl. By reaction volume is intended the region of the channel in which reaction is performed. The length of the area of the specific binding member will generally be in the range of about 10 nm to 5 cm, more usually 100 nm to 2.5 cm, frequently 10 microns to 10 mm, depending on the purpose of the operation and the required capacity for binding.

The substrate in which the capillary channels are formed may be of any convenient material, such as glass, plastic, silicon, or the like. Various plastic or organic polymeric materials include addition and condensation polymers and copolymers, linear or cross-linked, clear, semi-translucent, or opaque, mixtures of polymers, laminates and combinations thereof. Polymeric materials include polyethylene, polypropylene, acrylics, e.g. poly(methyl methacrylate), polycarbonate, poly(vinyl ethers), polyurethanes, dimethyl siloxanes, poly(4-methylpentene-1), etc. Desirably the polymers should be capable of extrusion or molding. Where the reaction sites are viewed directly, i.e., in situ, the covering in the device must be optically clear at the detection wavelengths employed.

Methods of fabricating channels in such substrates, and welding substrate and covering components are well known in the microfabrication field. It should be noted that localized or low-temperature welding techniques must be employed where the channel regions are initially loaded with a heat-sensitive biological material, such as a biological polymer or heat unstable binding agent. To this end, a variety of adhesives or techniques for surface-localized thermal binding are available, such as ultrasonic welding or laser welding.

FIGS. 2A and 2B are plan and sectional views, respectively of a multi-site reaction device 28 constructed in accordance with another embodiment of the invention. The device includes a substrate 30 and covering 32 which together, form a planar channel 34 in communication with input and output ports 42, 44, respectively. That is, the channel is a thin planar expanse formed between confronting surfaces 45, 47 of the substrate and covering, respectively. Bulk-phase liquid is moved in and out of the channel through the two ports.

As seen particularly in FIG. 2A, the planar channels includes a plurality of discrete reaction regions, such as regions 36, 38, 40 which are arranged in a two-dimension array of sites within the channel. Each reaction region, such as region 36, is defined by upper and lower wall portions, such as wall portions 36a, 36b, having a reaction-specific reagent bound thereto, preferably releasably, for release in the reaction region between the two wall portions, when bulk-phase medium is added to the channel. Exemplary modes of releasably binding reagents to a reaction site wall portion are discussed below with reference to FIGS. 7 and 8.

The distance d₁ between the confronting channel surfaces is between about 20-1,000 microns, preferably 50-500 microns. In particular, the channel thickness is dimensioned to substantially prevent convective fluid flow among the reaction regions when a bulk-phase liquid is introduced into the channel. In addition, the channel may be provided by porous barriers, not shown, that act to limit lateral convective flow. Such barriers may, for example, effectively partition the planar channel into a plurality of elongate subchannels, such as the subchannel aligned with ports 42, 44, and containing reaction regions 36, 38, where the distance between adjacent barriers is, for example, comparable to the channel width dimension in device 12.

FIG. 1B is a plan view of a multi-site reaction device 46 constructed according to another embodiment of the invention. The device is formed of a substrate 48 and covering 50 which together define a closed elongate channel 52 connected at its opposite ends to ports 58, 60, similar to device 12. The device differs from device 12 in that the reaction regions, such as regions 54, 56, formed within and along the length of channel 52, are radially enlarged. Preferably the reaction regions are shaped as in FIG. 1B to promote efficient removal of reaction-region components, e.g., products, from the device upon completion of the reactions in the device. The reaction sites contain reaction-specific reagent(s) releasably bound to wall portions of the regions, as above.

The depth d₁ and width d₂ dimensions in the device are similar to those in device 12, that is, preferably between 20 and 1,000 microns, more preferably between 50-500 microns. The lateral dimension d₃ of each reaction region is typically 1.5-3 times that of width d₂. This configuration has the advantage over device 12 in providing greater-volume reaction regions while still limiting convective flow between the regions through the narrowed connecting channel portions.

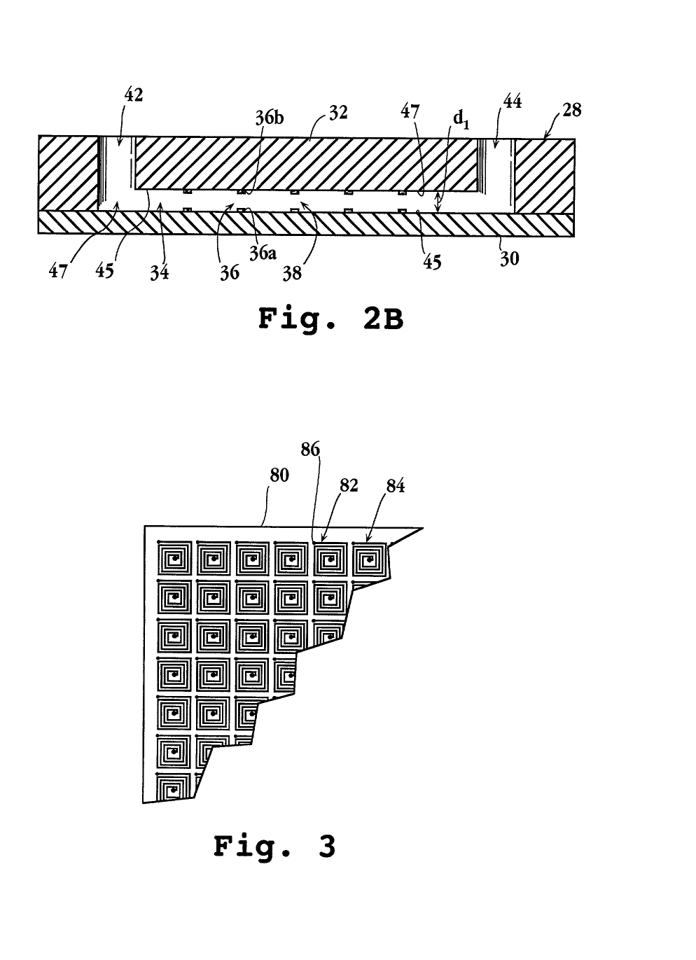

FIG. 3 illustrates a microfluidics card 80 which is formed to include a plurality of multi-site reaction site devices, such as devices 82, 84 of the type described above. Specifically, each device includes an elongate channel, such as channel 86 in device 82, and each channel includes a plurality of reaction regions within the channel and spaced along the length of the channel. The card illustrated, which includes and 8×12 array of devices, is designed for use in carrying out groups of up 96 simultaneous reactions, e.g., PCR reactions.

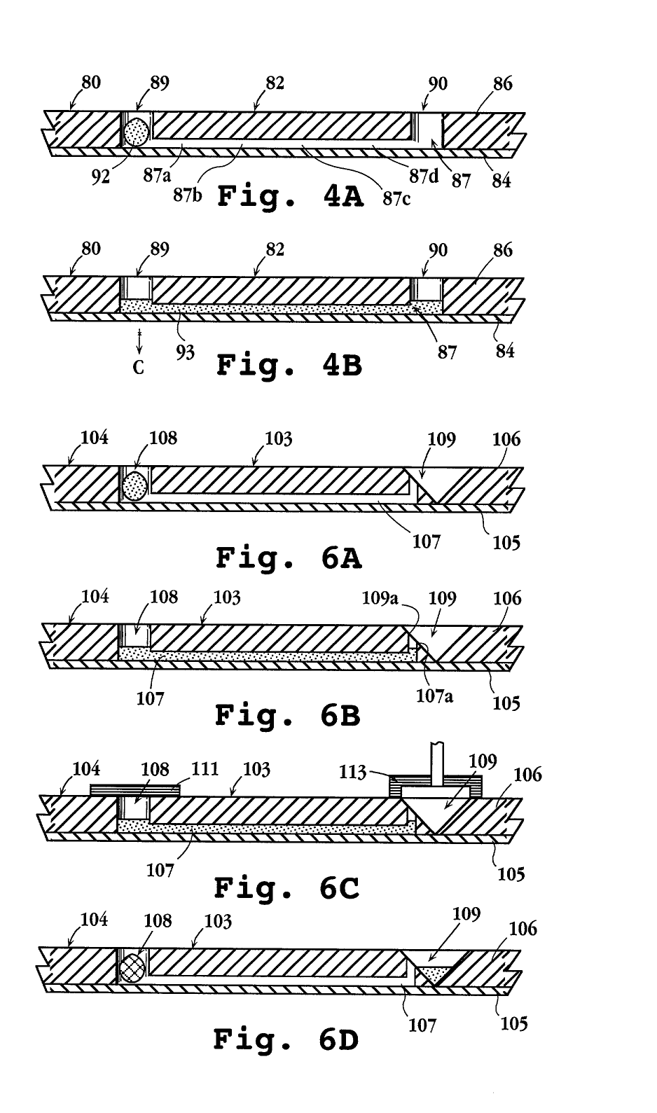

The construction of device 82 in card 80 is seen cross-sectionally in FIGS. 4A and 4B. The card includes a substrate 84 and a covering 86 which together define the spiral channel of each of the several devices in the card. Device 82, which is representative includes elongate serpentine channel 87 having inlet and outlet ports 88, 90 at opposite ends of the channel, and a plurality of reactions regions, such as regions 87a, 87b, 87c, and 87d within and along the channel. As above, each of the reaction regions carries reaction-specific reagent(s) releasably bound to the wall portion of that region. (The channel is shown in linear form in FIGS. 6-8, it being recognized that the inlet port is at one corner of the device, and the outlet port, at the center of the device, as in FIG. 3).

In FIG. 4A, a drop 92 of bulk-phase medium is placed in port 89 (and in the ports of other devices on the card). The card is placed in the bucket of a centrifuge subject to a centripetal force in the direction of arrow C, forcing the liquid droplet through the channel, as illustrated in FIG. 4B. The movement of liquid under the centrifugal field is self-limiting once a common liquid level is reached through the channel, since there is no longer a driving force on the liquid at this point. The sheet of bulk-phase liquid in the channel is indicated at 93 in FIG. 4B.

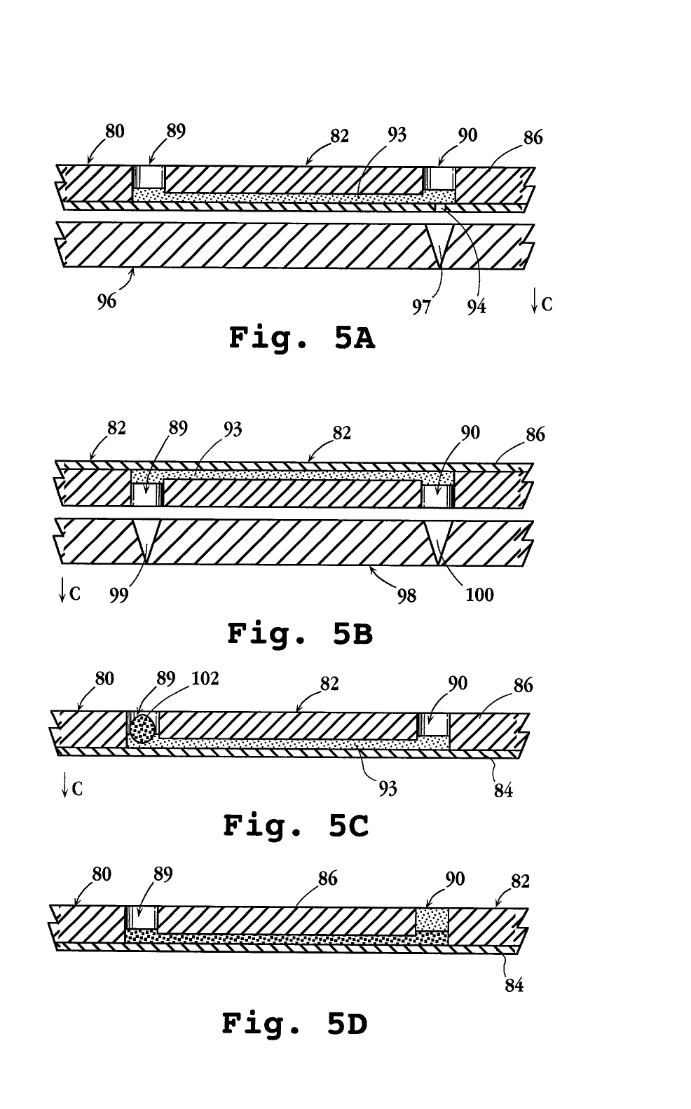

After carrying out the multiple simultaneous reactions in each device of the card, e.g., by successive heating and cooling in the case of a PCR reaction, the liquid in the device channels is removed for product analysis. Several liquid-retrieval methods are illustrated in FIGS. 5A-5D. In the method illustrated in FIG. 5A, the substrate is punctured, as at 94, at each of the device outlet ports, such as port 90 in device 80, and a capture plate 96 is placed against the substrate. The capture plate has a plurality of wells, such as well 97 which are arrayed on the plate for registration with corresponding outlet ports in the card devices. The card and capture plate are then centrifuged so as generate a force in the direction of arrow C in FIG. 5A, to drive liquid in each channel in the card into a corresponding well in the capture plate. The liquid samples in each well can then be individually handled by conventional microtiter plate methods.

Alternatively, and with reference to FIG. 5B, a capture plate 98 having wells, such as wells 99, 100 corresponding to the two ports in each device may be placed against the covering in the device, that is, with the device inverted. Centrifugation in the direction generating a force C then drives the liquid from each device into the two wells of the capture plate.

Yet another liquid-retrieval approach is illustrated in FIGS. 5C and 5D. In this method, a droplet, such as droplet 102, of a liquid more dense than the bulk-phase solution in the each channel is placed in the inlet port of each device, such as port 89 of device 82. The card is then centrifuged with a force in the direction of arrow C, causing the heavier liquid to displace the bulk-phase liquid in the channel and drive the sample liquid into the outlet port of each device, such as port 90. The sample can then be analyzed and/or removed according to standard microtiter plate methods.

FIGS. 6A-6D show an alternative construction of the devices, such as device 103 in a multi-device card 104. The card has the general construction of that described above, being formed of a substrate 105 and a covering 106 defining, and defining a plurality of multi-site reaction devices, such as device 103, in the card. Device 103, which is representative, includes an elongate spiral channel 107, having a plurality of reaction regions formed within the channel and spaced along its length, and inlet and outlet ports 108, 109, respectively. As seen in the figures, outlet port 109 communicates with and “upwardly” directed end portion 107a of the channel along an angled wall portion 109a thereof, such that the channel empties into an upper or distal portion of the port.

In operation, a drop of bulk-phase medium is placed in the inlet port of each device, such as port 108 in device 103, and the card is centrifuged, as described above, to force liquid into the channel, as in FIG. 6B. After carrying out multiple reactions in each of the loaded devices, bulk-phase medium is retrieved by (i) placing a seal 111 over each inlet port, such as inlet port 108 in device 103, and a suction device 113 over each outlet port, to draw liquid out of each channel and into the associated outlet port, as illustrated in FIGS. 6C and 6D, producing a sample of bulk-phase medium in each outlet port. The sample can then be handled according to standard microtiter plate procedures.

It will be appreciated that the various methods just described for introducing bulk-phase medium into a device channel, and removing it therefrom are also applicable to reaction system having a single device of the type described with respect to FIGS. 1 and 2.

Each reaction region in the device of the invention may have reaction-specific reagents(s) releasably bound to a wall portion that defines the region. By this is meant the reagent(s) remain anchored to the reaction site walls upon introduction of bulk-phase medium into the channel, but are released passively or actively thereafter, to participate in solution phase with a reaction in the reaction site. Alternatively, the region-specific reagent may be non-releasably bound to the wall portion, e.g., covalently bound, where the reagent can participate in the desired reaction in bound form. An example would be a bound DNA primer that can participate in primer-dependent DNA polymerization reaction.

The reagent may be any compound capable of participating in a biological of chemical reaction, and in particular, capable of reacting with one or more reactants in a bulk-phase medium to produce a reaction that is unique to the reaction region which contains the reagent. Thus, for example, the reagent may be one of a number of different binding agents or drugs, some or all of which are capable of interacting with a receptor carried in the bulk-phase solution, or one of a number of different enzyme substrates, some or all of which are capable of interacting with an enzyme contained in the bulk phase solution, or conversely, one of a number of different proteins or other enzymic or binding agents, some or all of which are capable of reacting with a given substrate or binding agent in the bulk-phase medium. In one preferred embodiment, detailed below, the reagent includes one or more oligo- or poly-nucleotides having a reaction-specific nucleic acid sequence effective to produce a sequence-specific reaction, such as one involving complementary strand hybridization or sequence-specific endonuclease cutting.

Desirably at each site there will be at least about 10 aftomoles, preferably at least about 1 femtomole, usually at least about 1 picomole and not more than about 1 millimole, more frequently not more than about 0.5 millimole of a specific binding pair member. The amount of the releasable reagent will depend upon the nature of the reaction, the specificity of the reaction, the signal produced, the sensitivity of the detection system, and the volume of the reaction region.

Depending on the nature of the surface of the channel, proteins or other substances may bind non-covalently and be stably bound during the operation. For example, methylated proteins strongly adhere to surfaces. The protein also serves to minimize non-specific binding of components of the operation. Alternatively, the reagent may be embedded in a wall coating, such as a hydrogel wall coating, or other coating material that hydrates or dissolves over a time period that is substantially greater than the time period needed to fill the channel in the device. Polymer coatings capable of holding and releasing reagents over time are also suitable for certain reagents.

Several methods of reagent binding for active release are also available. FIG. 7A shows a reaction region 130 with wall portion 132. Reagent molecules 134 are releasably bound to the wall portion by a linker covalently attached to the wall portion, and containing a photolytic group 136 that is cleaved by irradiation with a selected wavelength light, e.g., UV light. The design and synthesis of bifunctional reagents containing an internal photolytic group and capable of covalent attachment to active wall-portion functionalities, such as carboxy, amino, hydroxy or thiol groups, and to suitable reagent molecules are well known to those in the art. The reagent molecules are actively released, after addition of bulk-phase solution to the channel, by irradiating the channel with light of a photolytic wavelength.

In the method illustrated in FIG. 7B, the wall portion 142 in device 140 is covalently derivatized with streptavidin molecules 143, using well-known methods. A biotinylated reagent 144, such as biotinylated nucleic acid, is bound to the streptavidin through biotin groups, such as 146 attached to the reagent. If lower affinity binding is needed, the streptavidin may be replaced by lower-affinity binding agents, such as antibodies or receptors, and the biotin, by lower-affinity ligands, such as antigen or receptors binding agents. Release of the reagent from the wall portion can be effected, after introducing the bulk-phase medium, by application of heat or sonic energy, or another ligand that has a higher affinity for the binding agent.

FIG. 7C illustrates reagent binding through an enzyme cleavable linkage, in this case, an esterase. The figure shows a segment of a device 150 having a wall portion 152 and reagent molecules 154 covalently attached to the wall portion through ester linkages 156. Inclusion of an esterase in the bulk-phase medium, leads to slow passive release of reagent into the solution phase in the reaction region. Alternatively, in a device like the one described in Section IV below, the cleaving enzyme can be introduced into each reaction region from one of associated side channels, to actively release the reagent. Where the reagent is an oligo- or polynucleotide covalent bound the wall portion, the reagent may include a restriction-endonuclease site, for release from the wall portion by including the appropriate endonuclease.

In each of the attachment schemes described above, the site-specific reagent is attached to the wall portion by a common linkage or attachment to an immobilized molecule. That is, the linkage itself is common to all of the reaction regions. In this general case, the specific reagents must be added directly to specific reaction regions, either before the channel is covered or, as in the device described in Section IV below, by using the supply reservoirs to deliver a specific reaction region to each associated region.

In still another embodiment, the reagents are non-releasably bound to the reaction region wall portions, e.g., by covalent binding, and are employed in the reaction in immobilized form, e.g., immobilized nucleic acid primers sued in a DNA sequence reaction.

In another general case, and in accordance with one aspect of the invention, the particular reaction-specific reagents are designed to react with and bind to immobilized molecules that are unique to each reaction site. By this method, the device can be “programmed” with the releasable reagents simply by adding a mixture of the reagents to the channel, and allowing each reaction-specific reagent to bind to its binding pair in a selected reaction region.

The latter method is illustrated in FIG. 8, which shows three reaction regions 112, 114, and 116 in the channel of a device 110, where the corresponding wall portions are indicated at 118, 120, and 122, respectively. Covalently attached to each wall portion is a unique (site-specific) capture nucleic acid, such as oligonucleotide 124 (Si) attached to wall portion 118, and oligonucleotides S₂ and S₃ attached to wall portions 120, 122, respectively. The capture nucleic acids in the different region are preferably at least about 7-10 bases long, typically 12 bases or more, and differ from one another in sequence by at least one, and preferably two or more bases. The capture reagent may, in addition, contain more than one capture sequence, allowing different-sequence reagents to be captured on a single capture nucleic acid. The reagent itself, such as reagent 126 (P₁) in reaction region 112, has a capture portion 126a that is complementary in base sequence to the capture nucleic acid, and a reaction portion 126b which is effective to participate in the solution-phase reaction in the reaction region. Similarly, each of the reagents P2 and P3 in regions 114, 116, respectively, has a capture portion that hybridizes to capture nucleic acids S₂ and S₃, respectively, and a reaction portion that is unique to that reaction region.

In preparing the device with the different nucleic acid reagents, a bulk-phase medium containing a mixture of the reagents is circulated through the channel under hybridization conditions, for a period sufficient to saturate the capture nucleic acids in each reaction region with the different-sequence reagents.

III. Multiple-Site Reaction Method

The invention may be used with various protocols involving nucleic acid sequencing, nucleic acid hybridization, and the like, single nucleotide polymorphism (snp) detection, proteomics (protein-protein interactions), specific binding pair reaction (ligand-receptor), enzyme reactions, and the like. More generally, the invention may be used for any system that permits multiple reactions involving one or more common reactants, supplied to each reaction region in a bulk-phase medium, and one or more reaction-specific reagents that are supplied by each individual region.

In performing the operations, the temperature of the regions may be varied, by heating and cooling, using heating elements in contact with the region, infra-red sources or other sources of electromagnetic radiation, the pressure may be varied, the regions may be irradiated with light in the wavelength range of from about 200 to 2000 nm, and the like. Depending on the operation, heating and/or cooling may be desired, as illustrated by thermal cycling with PCR.

By way of example, several types of nucleic acid reactions can be carried out with the device of the invention. By having a main trench or channel, one has numerous sites with individual sources, so that at each site, the primers may be the same or different. A DNA sample is introduced into the main channel. The sample may be genomic DNA, a cDNA sample, a sample in which DNA fragments have been amplified using the polymerase chain reaction (PCR), genomic fragments, e.g. restriction endonuclease fragments, and other types of DNA sample material with a plurality of target sequences.

The sample is introduced to the site as single stranded DNA or may be denatured at the site, followed by reducing the temperature to provide for hybridization conditions. The hybridization medium is incubated for sufficient time for hybridization to occur between homologous or complementary sequences between the primer and the sample DNA, depending on the degree of stringency.

Where the device used does not contain supply reservoirs (the embodiments described in Section IV) the bulk phase medium added to the channel includes, in addition to the DNA or RNA sample material, common components required for the desired reaction, except for the reaction-specific oligo- or polynucleotides that will be provided in each reaction region. For example, for conducting simultaneous PCR reactions, the bulk-phase medium will contain, in addition to the DNA sample, a template-dependent polymerase, e.g., TAQ polymerase, all four deoxynucleotide triphosphates (dNTPs) and suitable salt and buffer components. In some instances one may have one, some or all four ddNTPs, or limiting concentration of some of the dNTPs, in the medium to provide termination at different nucleotide positions. Where the reaction is designed for primer extension, e.g., in DNA sequencing, the bulk-phase medium would contain mixtures of ddNTPs having a specific fluorescent species to designate each of the ddNTPs. Components employed in other nucleic acid reactions are considered below.

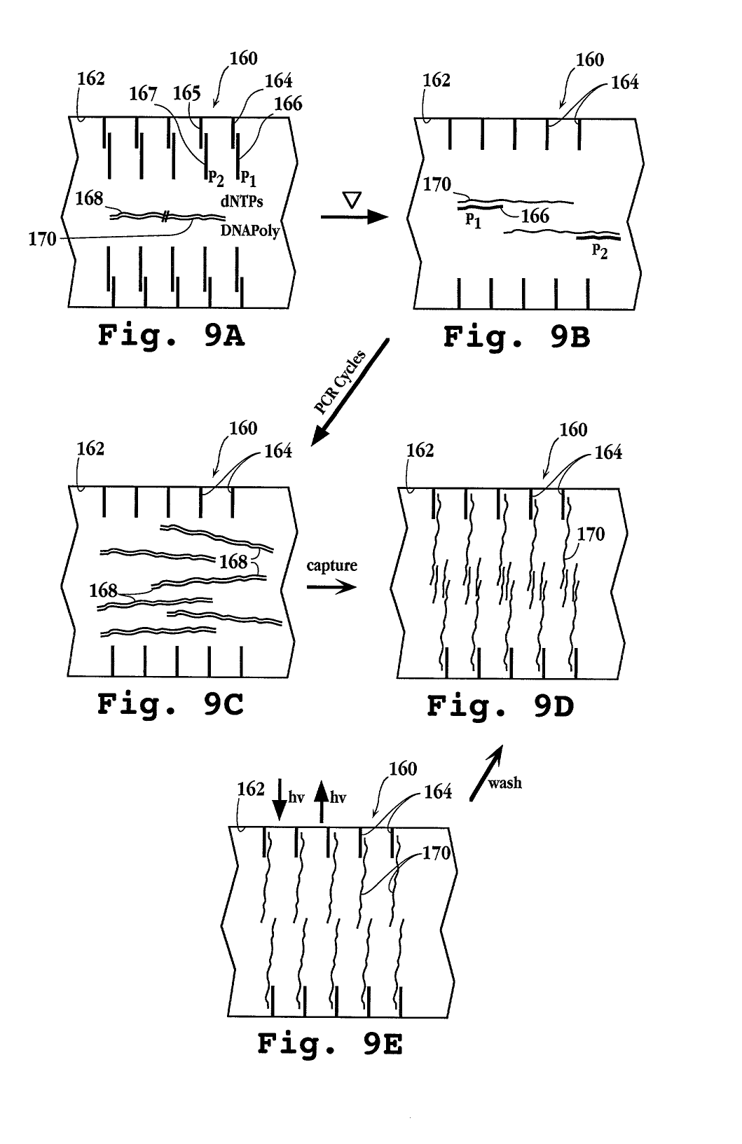

FIGS. 9A-9E illustrate steps involved in the use of the present invention for carrying out simultaneous PCR reactions. The figures show one reaction region 160 in a multi-channel device like the one shown in FIG. 1. The region has a wall portion 162 having covalently bound thereto, two different-sequence capture probes, 164, 165, which have sequences complementary to PCR primers 166, 167, respectively. The two primers (P₁ and P₂ in the figures) are reaction-specific PCR primers for a particular target DNA sequence, and are unique to reaction region 160. That is, different reaction regions includes a different set of PCR primers for amplifying a different target DBA sequence, it being recognized that some regions may have identical primers for control and sample duplicate purposes, or different quantities of the same primer sets.

A bulk-phase medium introduced into the device's channel includes double-stranded target DNA whose individual strands are indicated at 170. The bulk-phase medium also includes other PCR reaction components as noted above. The device is heated to DNA denaturing temperature, simultaneously denaturing the sample dsDNA and releasing primers P₁ and P₂ from the wall portion in each reaction region, as indicated in FIG. 9B, which also shows the primers annealed to the sample single strands after cooling under annealing conditions. The heating step typically is such as to raise the temperature of the bulk-phase medium to about 94° C. for a period of 1-5 minutes.

After a selected number of cycles of heating and cooling to effect denaturation, annealing and extension, the reaction mixture in each reaction region includes amplified sample dsDNA product or amplicon, as indicated at 168 in FIG. 9C, where the amplicon is different for different regions.

In one embodiment of the method, the bulk-phase medium is removed from the channel, yielding a mixture of all of the individual amplicons that can then be individually analyzed and/or isolated, e.g., by gel electrophoretic methods.

Alternatively, in a second embodiment, each channel can be employed as an electrophoretic separation channel, by applying a voltage potential across the channel ports, and detecting and/or isolating each amplicon as it migration past a detection and/or collection point adjacent one of the ports.

In a third embodiment, illustrated in FIG. 9D, the amplicons are partially purified by capture in single-stranded form on the capture probes in each reaction regions, by a capture heating and cooling step, and flushing the channel to remove unbound material. It will be appreciated that in this embodiment, the capture probes must contain sequence complementary to a sequence in the amplicons, and preferably to each amplicon strand.

In a fourth embodiment, illustrated in FIGS. 9D and 9E, the amplicons are both captured within each associated reaction region, and analyzed in situ in captured form. In this embodiment, the PCR reaction is carried out in the presence of detectable probes, such as fluorescently labeled nucleotides. The amplicon strands are optionally captured on the capture nucleic acids, and analyzed in situ, e.g., by examining each reaction region successively with a fluorescence scanner or microscope, to determine the presence and/or qualitative amount of fluorescence present in each reaction region.

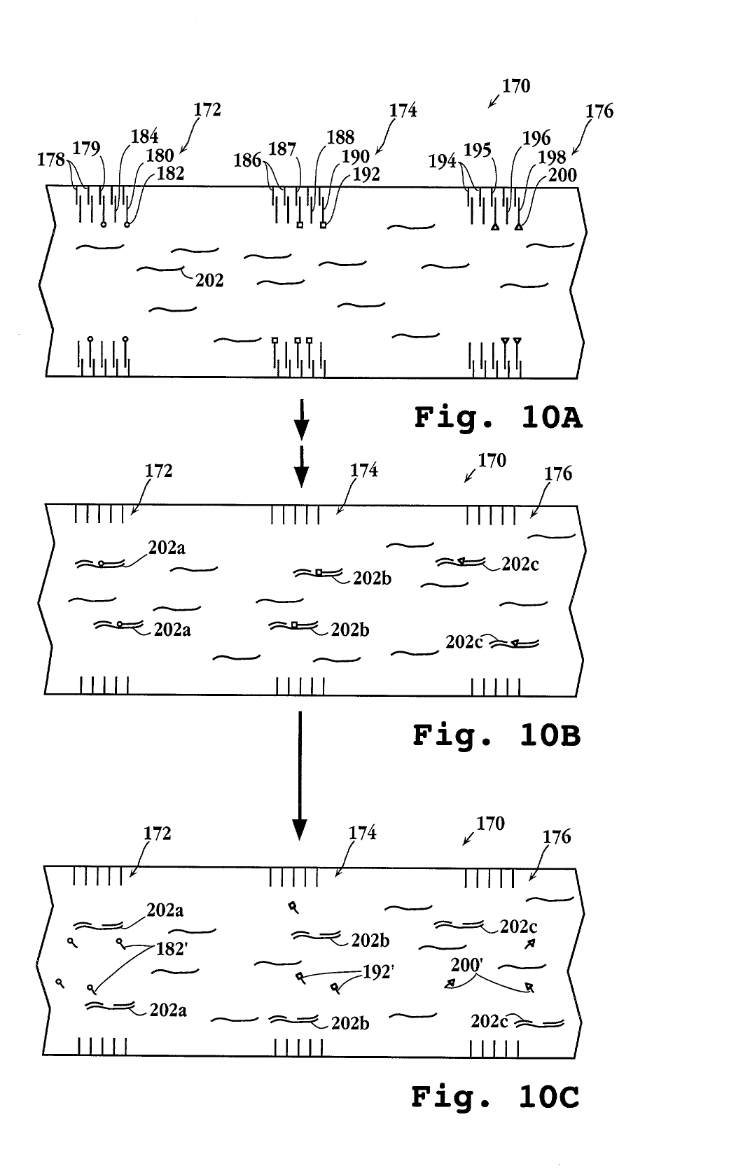

FIGS. 10A-10C illustrate a sequence analysis method that is advantageously carried out in accordance with the present invention. The method employs DNA primers having 5′-end electrophoretic tags that having (i) unique electrophoretic mobilities, by virtue of unique charge/mass ratio, and (ii) detectable moieties, such as fluorescent groups. Such tags are detailed, for example, in co-owned patent applications Ser. No. 09/303,029, filed Apr. 30, 1999, Ser. No. 09/561,579, filed Apr. 28, 2000 and corresponding PCT application PCT US00/10501, Ser. No. 09/602,586, filed Jun. 21, 2000 and Ser. No. 09/684,386, filed Oct. 4, 2000, all of which are incorporated herein by reference.

The figures show three reaction regions 172, 174, 176 in a multi-reaction device 170. Each reaction region contain two different-sequence immobilized capture probes, such as probes 178, 179 in region 172, probes 187, 186 in region 174 and probes 194, 195 in region 176. In the particular method to be described, for detecting single base mutations, such as snps, in target DNA, the oligonucleotide reagents that are carried on and released from the capture probes include an unlabeled upstream primer, which is designed to bind the target DNA upstream of the site of mutation, whose binding to the target site is determined by the presence or absence of the potential mutation. The upstream primers include primer 184 in region 172, primer 188 in region 174, and primer 196 in region 176. The site-specific primer includes a detectable electrophoretic tag, such as described and referenced above, that can be used to provide a characteristic electrophoretic signature of that primer. In the figure, the site-specific primers and their detectable tags are indicated respectively at 180, 182 in region 172; at 190, 192, in region 174; and at 198, 200 in region 176.

In operation a bulk phase medium containing a plurality of target DNA 202, and a DNA polymerase with 5′-exonuclease activity is added to the device channel, bringing the target DNA and other bulk-phase reaction components, e.g., all five dNTPs, into each of the reaction regions, as illustrated in FIG. 10A. The device is then heated, or otherwise treated to release the two primers in each reaction region, and subsequently cooled, as above, to anneal the primers to upstream and mutations sites on region-specific target sites. The step is illustrated in FIG. 10B, where target DNA strands 202, 204, and 206 in the three regions indicate different target sequence that are complementary to the primers in the three different regions. In particular, the upstream primer will hybridize to a region upstream of a potential mutation in the specific target region, and the extent of binding of the site-specific primer to the mutation site target area will be influenced by the presence or absence of a particular base at the mutation site.

After primer binding to the respective target regions, the action of the polymerase enzyme begins to extend the upstream primer until the growing chain reaches the site-specific mutation. Depending on the presence or absence of a given base at the potential mutation site, the enzyme will cleave the electrophoretic tag from the site-specific primer, releasing it from the target/primer dsDNA, as indicated in FIG. 10C.

The bulk-phase medium may now be removed from the channel, as above, and the electrophoretic tags detected and identified by electrophoresis, thus to identify particular mutations contained in the target DNA. As above, the reaction products, particular cleaved and uncleaved site-specific primer sequences, can be recaptured within each reaction site, to remove such sequences from the bulk-phase sample before analysis.

In a variation of the method, the release of tags from the site-specific primers will be detected by (i) capturing all of the cleaved and uncleaved primer on the reaction-site wall portion, (ii) applying a potential difference across the two channel ports and (iii) sequentially detecting tags as they pass through a detection zone near the downstream end of the channel. In this method, the tags from the different primers will all have the same electrophoretic mobilities, so that the presence or absence of a tag in any reaction region can be determined by the absolute migration times of each detected tags, or the relative migration times of adjacent tags.

The method and device provide a number of advantages in carrying out simultaneous reactions involving nucleic acid targets. For carrying out simultaneous PCR reactions, the method minimizes the possibility of specious amplification products formed by mismatched primers, since each reaction is carried out substantially in the presence of one primer set only. The reaction in each region can be carried out to higher amplicon levels, since the concentration of a single primer pair in each region can be relatively high. Finally, the amplicon products can be detected directly in isolated form, by capture of labeled amplicon strands on the wall portion of each reaction region.

Similar advantages apply to DNA extension methods, such as the one described with respect to FIGS. 10A-10C. The possibility of false positives, due to primer mismatches, is substantially reduced because only a single primer pair is present in each reaction region (or only a single primer pair is present at high concentration, considering the possibility of some primer diffusion from adjacent reaction sites). The amount of signal produced can be enhanced, because of the greater concentration of a single primer or primer set in each reaction region. Finally, the reaction products can be detected in situ, by electrophoresis of reaction products through the device channel, or by analyzing individual reaction components in the bulk-phase solution.

The variation in reaction protocols can be expanded in a device like that described in Section IV below, having supply reservoirs feeding each reaction region in a device. For example, excess soluble primer sequence (unable to bind at the site to the surface) may be added under mildly denaturing conditions to displace the primer from the wall portion. Reaction products, such as labeled DNA or duplex DNA can be diverted from the channel directly into a side channel for detection in a side-channel reservoir. Restriction endonuclease or other site-specific reagents may also be introduced into the individual reaction regions in this embodiment of the device.

Assays that may be performed may be homogeneous (no separation step) or heterogeneous, requiring a separation step, although the detection may be at the channel site or a distal site. Assays may involve labels such as light emitting detectable labels, e.g. fluorescers, chemiluminescers, energy transfer labels involving two different dyes at a distance which results in energy transfer upon irradiation of one dye and emission of the other dye, lanthanide dyes, which provide time delayed emission, where the lanthanide dyes may be used in particles, since they do not result in significant energy transfer or quenching, etc., enzymes, where the substrate results in a detectable product, which can be a dye, fluorescer, radioisotope, particle, etc., radioisotope, particle, e.g. colloidal carbon, colloidal gold, latex, and the like.

For the heterogeneous assays, the protocols may involve release of the detectable label, so that the detectable label is assayed distal from the channel site. As illustrative of an assay would be the determination of a protease. By having a detectable label bound to the surface by a chain having a recognition sequence for the protease, one can monitor compounds modulating the activity of the protease. One may bind the detectable label through the proteolytically hydrolysable group to the surface at the site. One would premix the enzyme and the candidate compound to allow for binding of the two components. The mixture would then be moved through a lateral branch channel to the main channel site and allowed to incubate, ensuring that any additional reagents necessary for the proteolysis were present. After sufficient time for reaction to occur, the mixture at the main channel site would be moved into a lateral branch channel for detection of the label. The signal observed would then be related to the effect of the candidate compound on the enzyme activity. Rather than a candidate compound, there may be instances when one is interested in the enzyme activity of a cell. In this case a lysate could be prepared, where the enzyme of interest may be further processed to remove debris, other proteins, e.g. using HPLC, an affinity column, etc., and then moved through the lateral branch channel to the main channel site. Again, one could measure the activity of the enzyme in the lysate. Usually, one or more control may be performed in the same way as the assay, for comparison of the result from the sample.

There are numerous protocols for enzyme assays, depending upon the nature of the enzyme and the information desired. For example, one may be interested in a protease and/or proenzyme, where the protease activates the proenzyme. By binding the protease at the main channel site, one can add a sample suspected of containing the proenzyme to the main channel site and incubate for sufficient time for any proenzyme to be activated. One would then add substrate for the activated enzyme, where the product of the substrate can be detected. A similar assay could be to detect an enzyme requiring a coenzyme to form a holoenzyme.

Other assays may involve ligand-receptor binding, which may be competitive or non-competitive. In the competitive mode, a labeled ligand biomimetic would be non-covalently bound to the receptor, which in turn would be bound to the channel site. The ligand competitor would be moved to the channel site and allowed to incubate, where the degree of displacement of the labeled biomimetic would depend on the binding affinity of the ligand competitor. The binding affinity may be determined using kinetic or equilibrium measurement. This assay can be carried out homogeneously, where binding of the biomimetic to the receptor affects the signal, for example, fluorescence polarization or quenching. Quenching may be as a result of the interaction between the receptor and the label or the presence of a quencher bound to the receptor. By reading the change in fluorescence, one can determine the binding affinity of the ligand competitor. At completion of the assay, one would wash the site free of the released biomimetic ligand and the ligand competitor and then replenish the labeled biomimetic through the lateral channels. After washing any excess biomimetic ligand from the channel site, the channel site would be ready for the next assay.

In other assays one may use particles that provide for detection when the particles are in close proximity. One may use the LOCI technology, where one particle has a catalyst for forming singlet oxygen from hydrogen peroxide and the other particle has a dye that provides a detectable signal upon reaction with singlet oxygen. See, for example, U.S. Pat. Nos. 5,545,834 and 5,672,478. By having one of the pair of particles fixed at the main channel site and the other in solution, when the particles are brought together at the main channel site, in the presence of the other reagents, a signal will result. The effect of a candidate compound on the degree to which the particles are brought together can be a measure of the activity of the candidate compound. In any combination of two components that have a specific affinity for each other, the signal will be related to the degree to which the candidate compound interferes with the binding. Thus, one may interested in ligand receptor binding, whether a naturally occurring protein or candidate compound interferes with or augments complex formation between two proteins, the presence of a component in a sample in a diagnostic assay, where the component may be a drug, pollutant, pesticide, process contaminant, etc.

The assay would be performed by mixing the soluble particle with the compound to be assayed and moving the mixture with the additional reagents to the main channel site. The mixture would be incubated to allow for binding to occur and the signal read. The resulting signal could be compared with a control to determine the activity of the compound being assayed. As before, at completion of the reaction, the site could be washed free of all of the spent and unspent reagents and the process repeated.

Where one is interested in a polyepitopic compound, one has the opportunity to use non-competitive binding. For detecting the presence of a polyepitopic compound, one could use an ELISA assay, employing two antibodies: a bound antibody and a labeled antibody, where the two antibodies bind at different epitopes of the compound. One would add the compound through a lateral channel to the main channel site and incubate to allow for binding. One would then pass a wash solution through to remove non-specifically bound components of the sample, followed by addition of the labeled antibody. After washing away unbound labeled antibody, one would then detect the label present at the main channel site. The subject methodology may also be used to enrich a mixture for a desired component by providing for capillary electrophoresis in the source lateral channel, where the desired component would be concentrated when encountering the site. The remaining components could be washed through the site, where non-specific binding components would not be retained in the main channel, but directed to a waste channel.

The device may have independent supply and hold reservoirs, as described in Section IV below, or may have a connecting channel between multiple source and/or waste reservoirs, so that solutions may be added or withdrawn simultaneously from a plurality of reservoirs, may have crossed channels at the source for precise injection of volumes into the main channel, may have a plurality of reservoirs feeding into the source channel or receiving waste from the waste channel, etc. The waste channel may have a detector, providing means for irradiation of the waste channel and detection of light emission or absorption, or there may be a channel independent of the waste channel or incorporating a portion of the waste channel that serves as a detection channel. The solutions may be moved in any convenient way, pneumatically-positive or negative pressure, electrokinetically-electrophoretically or electro-osmotically, hydraulically, or the like. The choice will be based on accuracy, nature of the operation equipment available, sensitivity to variations in volumes, etc. Therefore, the reservoirs will be fitted with the necessary devices to provide for liquid movement.

IV. Multisite Reaction Device with Reaction-Region Reservoirs

In another aspect, the invention includes an apparatus having a multi-site reaction device provided with region-specific reservoirs for transferring material, e.g., solvent and/or solvent components, into or out of the individual reaction regions in the reaction channel. The region-specific reservoirs are generally of two types. A “supply” reservoir functions either to supply reagents or other reaction components from the supply reservoir to the associated reaction region, or serves as a source of liquid, e.g., electrolyte, for moving material in the corresponding reaction region out of the reaction region. A “hold” reservoir functions either to receive sample material from the associated reaction region or as a liquid drain when material is moved from the corresponding supply reservoir into the associated reaction region.

A reservoir may be a region-specific reservoir associated with a single reaction region, e.g., through a side channel connecting that reservoir to the reaction channel at or adjacent the corresponding reaction region, or the reservoir may be a common reservoir which services a plurality of reaction regions, e.g., through a plurality of side channels connecting the single reservoir to each of the reaction regions. As will be detailed below, the reservoir configurations are of four general types.

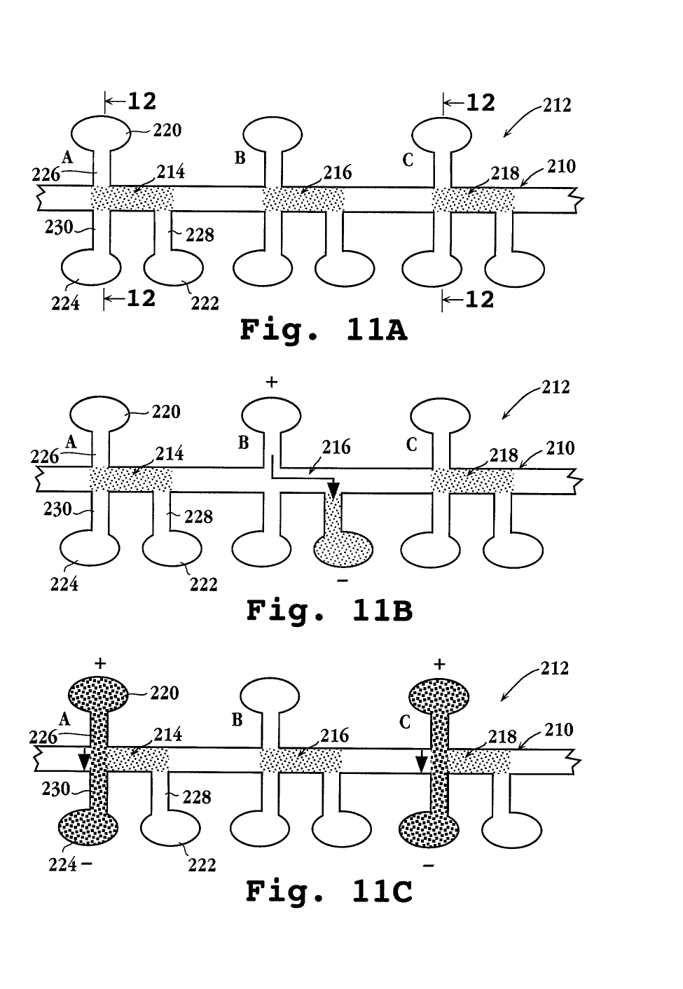

(A) a plurality of region-specific supply and hold reservoir pairs, where each pair is associated with an individual reaction region in a reaction channel. The reservoir “pairs” may include additional reservoirs, such as an additional hold reservoir, as illustrated in FIGS. 11A-11C;

(B) a common supply reservoir and a plurality of region-specific hold reservoirs in fluid communication with each of the reaction regions;

(C) a plurality of region-specific supply reservoirs and a common hold reservoir in fluid communication with each of the reaction regions, and

(D) a common supply reservoir and a common hold reservoir, both in fluid communication with each of the reaction regions.

The reaction-specific reagents employed in the region-specific reactions in the reaction channel may be carried on a wall portion of each reaction region, as described in Sections II and III above, or may be contained in the region-specific supply reservoirs associated with each reaction region.

Also included in the apparatus is transfer structure or means for transferring solvent or solvent components from or to a selected supply or hold reservoir, to or from the associated reaction region(s). As will be detailed below, the transfer means may be electrokinetic, e.g., liquid movement by electro-osmosis (EOF) or sample-component movement by electrophoretic movement, or pressure driven, effective to produce a fluid-pressure gradient across the reservoirs.

The transfer means are activated, for selective transfer of solvent or solvent components from or to the reservoirs, by a control unit whose construction and operation will be understood from the operation of the apparatus described below.

A. Device with Region-Specific Reservoirs

FIGS. 11A-11C show in plan view, a portion of a multisite reaction channel 210 in a device 212 constructed in accordance with one embodiment of the invention. In particular, the drawings show three reaction regions 214, 216, 218, in a reaction channel having a plurality, e.g. 5-10 or more such reaction chambers along its length. The dimensions of the reaction channel are like those described in Section II above. It is understood that some, but not necessarily all, of the reaction regions have associated pairs of supply and hold reservoirs, for transferring material in or out of the region.

Associated with each reaction region is a supply reservoir, such as reservoir 220 associated with region 214, and first and second hold reservoirs, such as reservoirs 222, 224, respectively, associated with the same region, and disposed on the side of the channel opposite the supply reservoir. Each reservoir communicates with the associated reaction region in the channel via a side channel, such as side channel 226 connecting supply reservoir 220 to the upstream side of region 214, side channel 228 connecting first hold reservoir 222 to the downstream side of region 214, and side channel 230 connecting second hold reservoir 224 to the upstream side of the same region. All three reservoirs are contain a liquid medium, e.g., electrolyte or non-electrolyte aqueous medium containing suitable buffer, salt, and/or chemical reagents, as will be appreciated from the operation of the device described below. Additional reservoirs, such as a second supply reservoir, may also be present.

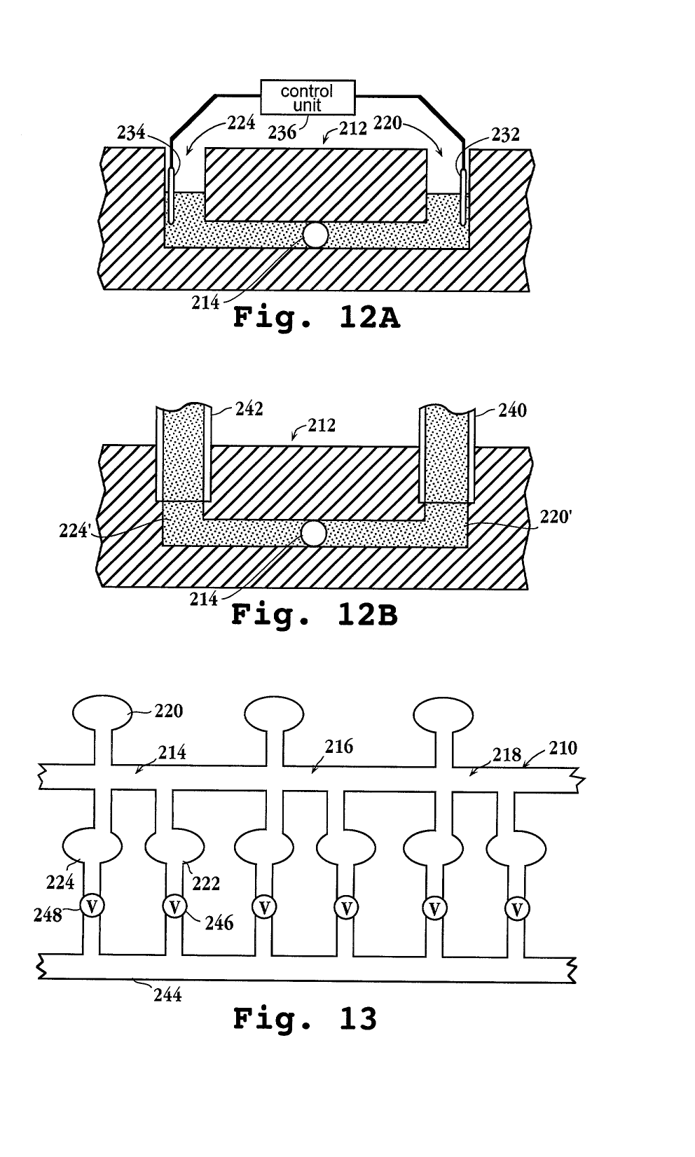

Exemplary transfer means or structure for transferring or moving medium and/or charged components in the medium from or to each reservoir, to or from the associated reaction medium are illustrated in FIGS. 12 and 13. FIGS. 12A and 12B are cross-sectional views of a channel region in device shown in FIGS. 11A-11C, taken along section line 12-12 in FIG. 12A. The embodiment illustrated in FIG. 12A includes open reservoirs 220, 224 adapted to contain a liquid medium, e.g., electrolyte, in contact with reservoir electrodes 232, 234, respectively, which are operatively connected to and under the control of the control unit 236 in the device. The control unit is operable to place a voltage potential across the two electrodes effective to move material from one reservoir into reaction channel 214, toward the other reservoir. As noted above, the electrokinetic movement may be bulk-phase flow by EOF or electrophoretic movement of charged component(s) in the reservoir medium or reaction region.

In the embodiment shown in FIG. 12B, the reservoirs, here denoted 220′ and 224′, are ports adapted to removably receive capillary tubes, such as tubes 240, 242, respectively, which form part of the reservoirs, allowing the reservoir material to be readily replaced during an operation, by replacement of the capillary tubes and the material contained therein. The transfer means associated with the capillary tubes may be either electrodes for applying a voltage potential across the tubes, or a source of pressurized fluid, for creating a fluid pressure across the tubes.

The latter transfer means is illustrated in FIG. 13 which shows the channel and reservoir configuration seen in FIGS. 12A-12C operatively connected to a channel 244 which in turn is connected to a reduced-pressure or vacuum source through a series of valves, such as valves 248, 246 connecting hold reservoirs 224, 222, respectively to the channel. The valves are operatively connected to the control unit for switching between open and closed valve conditions. Microvalves suitable for use in a microfluidics device are well known. As can be appreciated from FIG. 13, opening of any valve, such as valve 246, promotes flow of liquid from the corresponding supply reservoir through the associated reaction region, such as region 214, toward the associated hold reservoir, such as reservoir 224. Other transfer means include electrokinetic, as above, pneumatic, e.g. pumping, hydraulic, piezoelectric, or sonic. The particular choice will depend upon convenience, the precision with which the solution must be metered, the volume of solution, the nature of the equipment, i.e. the capabilities of the equipment, and the like.

With reference again to FIGS. 11A-11C, the first of these figures shows reaction material (shaded) contained in each reaction region, corresponding roughly to the segment of the reaction channel between the upstream and downstream reservoir side channels, such as side channels 226 and 228 in region 214. In one exemplary transfer operation, it is desired to move the reaction material in reaction region 216 only out of the reaction channel, for example, for purposes of carrying out a further reaction on the components in the region, or for purposes of removing these components from other components contained in the channel. This is accomplished, as illustrated in FIG. 11B, by placing a voltage potential (or pressure-gradient) across the reaction region's supply reservoir and first hold reservoir, as indicated by the “+” and “−” signs in the figures, causing liquid flow into and through the reaction region, to transfer material in the reaction region into the corresponding second (downstream) hold reservoir as indicated.

A second general type of transfer, illustrated in FIG. 11C, involves the transfer of liquid or liquid components through the aligned side channels connecting a supply reservoir and the second (upstream) hold reservoir to the reaction channel. In one application, this transfer is used to introduce one or more reaction reagents, which can be region-specific reagents, into each of the reaction regions. This application is illustrated in the figure, which shows the introduction of reagent material (cross-hatching) from the first the third supply reservoirs in the figure into the upstream portion of reaction regions 214 and 218, respectively. The reagent material, being substantially localized in each corresponding reaction region, is effective to promote region-specific reactions with the components contained in each reaction region. This mode of reagent transfer can be used, in other words, to replace the delivery of region-specific reagents via the wall portion of each reaction region, as described in Section II. It can also be used to introduce new reagents selectively into one or more reaction regions during operation, or to sample a portion of the reaction volume in one or more selected regions, by transferring a portion of the reaction volume into the corresponding second (upstream) hold reservoir.

In a typical operation of the device, the reservoirs and side channel are preloaded with the desired solution, e.g., by placing the solution in an open reservoir or adding the solution via a capillary reservoir. The solution will be drawn into the associated side channel by capillarity. When the solution reach the junction of the side channel and reaction channel, it may continue to flow into the reaction channel, or more preferably, will stop at the side-channel junction due to the abrupt channel in channel dimension, or because of a hydrophobic barrier placed at the junction region, e.g., around the junction end of the side channel. If it is desired to have liquid continue to flow into the reaction channel, the junction interface can be smoothed to present a continuously smooth junction.

After filling the reservoirs, the bulk-phase liquid is introduced into the channel, e.g., by one of the methods described in Section II above. The introduction of bulk-phase material may displace reservoir material already in the channel, or may simply coalesce with the reservoir solutions at the side-channel interfaces. Following introduction of the bulk-phase material and, if necessary, introduction of region-specific reagents into the regions, the region-specific reactions are allowed to proceed. Following this, additional transfer steps involving movement of reactant material in or out of the reaction regions and hold reservoirs may be executed, as now will be outlined.

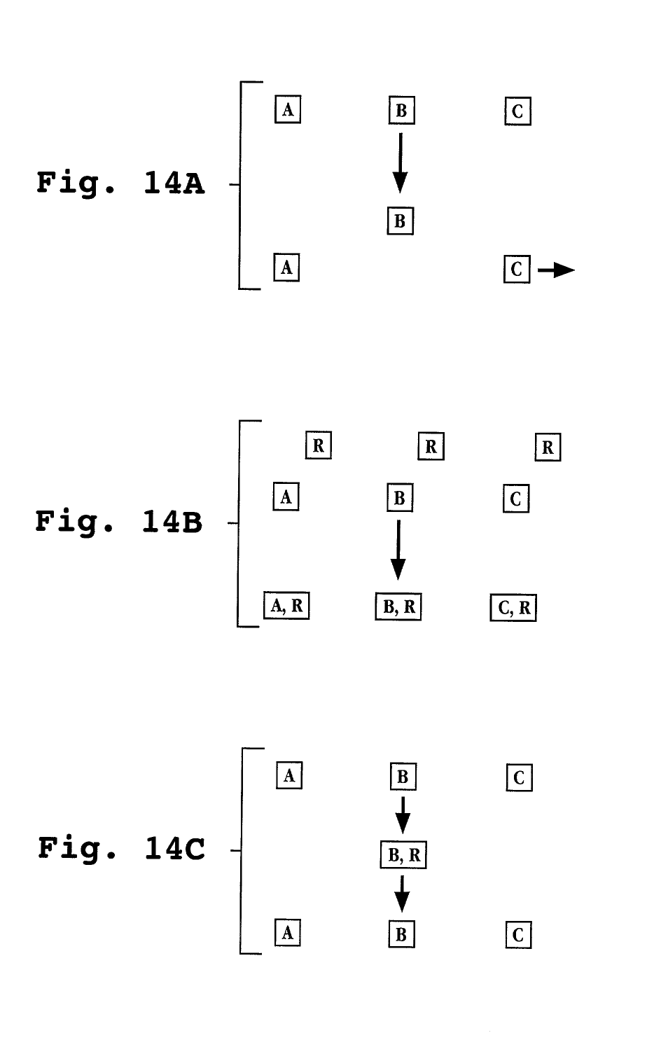

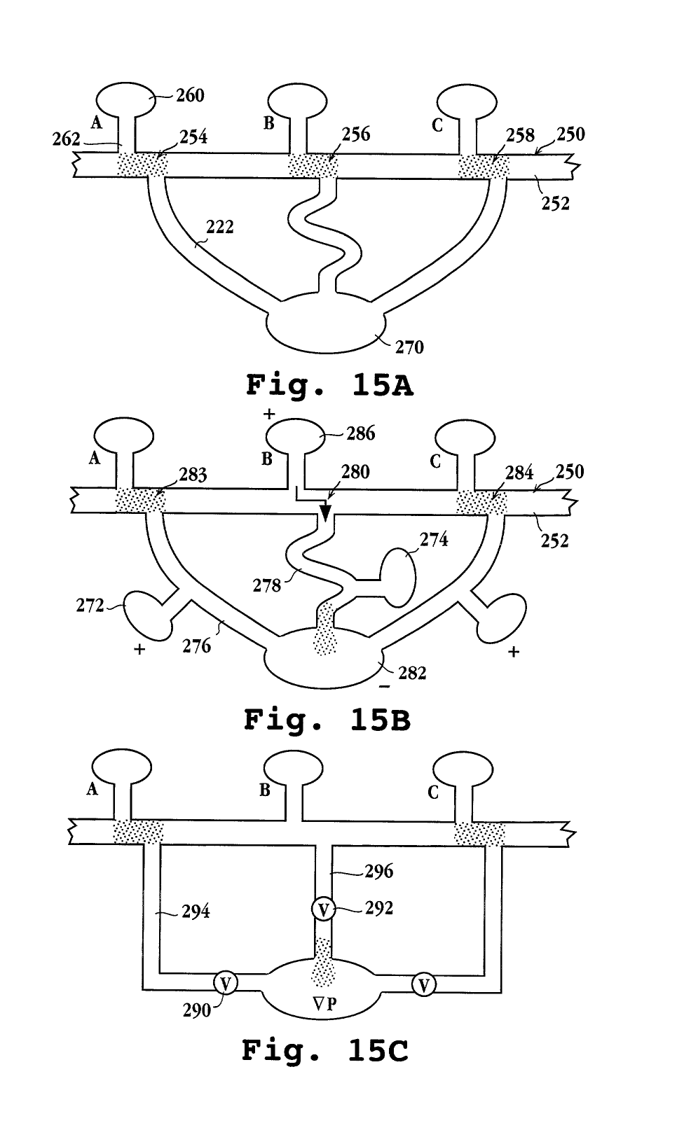

FIGS. 14A-14C illustrate various sample- or reagent-transfer operations that are possible with the region-specific reservoir configuration just discussed. In FIG. 14A, the sample material B from the second region is selectively removed into the corresponding first hold reservoir, allowing remaining components A and C in the first and third regions to be removed (and later combined) in the reaction channel. Alternatively, the transferred material B might be assayed, and depending on the results of the assay, either returned to its original region, or transferred to another station for further processing.

FIG. 14B illustrates the addition of a common reagent R to each of the reaction regions, by transferring the reagent into the reaction regions through the reservoirs with aligned side channels associated with each region. As indicated, this brings reagent R into reaction proximity to each of separated components A, B, and C. Alternatively, and as mentioned above, the reagent introduced into each region may be region-specific.

Various other permutations of these operations are also easily achieved. For example, FIG. 14C illustrates a two-step transfer operation in which component B is first moved to a first hold reaction, reacted there with reagent R, to produce component B′, then returned to the original reaction region in the channel.

B. Device with Both Region-Specific and Common Reservoirs

In another general embodiment, the device of the invention has a common supply reservoir and a plurality of region-specific hold reservoirs in fluid communication with each of the reaction regions; or a plurality of region-specific supply reservoirs and a common hold reservoir in fluid communication with each of the reaction regions. By “common reservoir” is meant that at least some of the reaction regions in the reaction channel are connected to a single supply or hold reservoir via side channels, it being understood that some other of the reaction regions in the reaction channel may be serviced by pairs of region-specific supply and hold reservoirs, and other regions may not have associated reservoirs of either type.

FIGS. 15A-15C illustrate portions of a reaction channel region in three different embodiments of the invention in which the reservoir configuration is region-specific supply reservoirs and a common hold reservoir. The first embodiment, indicated at 250 in FIG. 15A, includes a reaction channel 252 with a plurality of reaction regions, including regions 254, 256, and 258, shown. Each reaction region has an associated supply reservoir, such as reservoir 260 associated with region 254, connected to the channel through a side channel, such as side channel 262. The three reaction regions (and others in the reaction channel) are each connected to a common hold reservoir 270 through side channels, such as channel 266 connecting the common reservoir to reaction region 254.

In the embodiment of FIG. 15A, liquid flow from the plural reaction regions to the common hold reservoir is passive, that is, in the absence of region-specific control elements, such as those described below with respect to FIGS. 15B and 15C. This first embodiment would be appropriate, for example, for introducing different reaction reagents simultaneously into each of the different reaction regions. In order to ensure uniform flow characteristics during such simultaneous liquid movement, the flow resistance between each supply reservoir and the common hold reservoir should be substantially the same for each reaction region. Flow resistance is determined largely by the cross-sectional area and total length of the side channels. Assuming a given channel cross-sectional area, and standard dimensions in the supply-reservoir side channels, the device is preferably constructed such that each of the hold-reservoir side channels, such as channel 226, have the same overall length. As indicated, this may require that one of more of the side channels be given a serpentine or otherwise curved shape. The transfer means in this embodiment may be either electrodes for establishing potential differences across the reservoirs, or means for channel structure for placing a pressure differential across the reservoirs.

The device of FIGS. 15B and 15C are like that above, except that in both embodiments, each hold-reservoir side channel has a control element for controlling movement of liquid or sample components through the side channel. In the embodiment of FIG. 15B, the control elements are reservoirs associated with the side channels, such as reservoirs 272, 274 associated with side channels 276, 178, respectively. These reservoirs, like the supply and common hold reservoirs in the device, are provided with electrodes for applying selected voltage potentials across selected pairs of reservoirs, under the control of a control unit that contains a suitable voltage source.

In the example illustrated in FIG. 15B, assume it is desired to selectively move reaction material from center reaction region 280 into common hold reservoir 282, without moving the reaction material in adjacent reservoirs 283 and 284 from the reaction channel. This is done, under the control of the above control unit, by applying a voltage potential across supply reservoir 286 and common hold reservoir 282. Since this also places a similar voltage potential across all of the reactions regions and the common electrode, it is necessary to “neutral” this potential by placing a voltage potential on all non-selected hold-reservoir side channels. This is accomplished, in the present embodiment, by placing the non-selected side channels at substantially the same voltage as the reaction channel voltage. The result is sample flow from reaction region 286 to reservoir 288 only. This voltage configuration can be timed to permit sample flow into the hold reservoir, without continue flow into the common hold reservoir from other side channels.

In the embodiment shown in FIG. 15C, the three reaction regions are serviced by region-specific supply reservoirs and a common hold reservoir under the control of a fluid-pressure gradient. The embodiment Includes, as control elements, valves such as valves 290, 292, that control the flow of liquid through each side channel, such as side channels 294, 296, respectively, connecting a reaction region to the common hold reservoir. Movement of liquid from one or more selected supply channels, through the corresponding reaction region and into the common hold reservoir is effected by placing a pressure differential across selected supply and hold reservoirs, and activating (opening) the associated valves. Since flow resistance in this embodiment is either “off” or “on”, it is less critical that the hold-reservoir supply channels be constructed to have equal flow resistance.

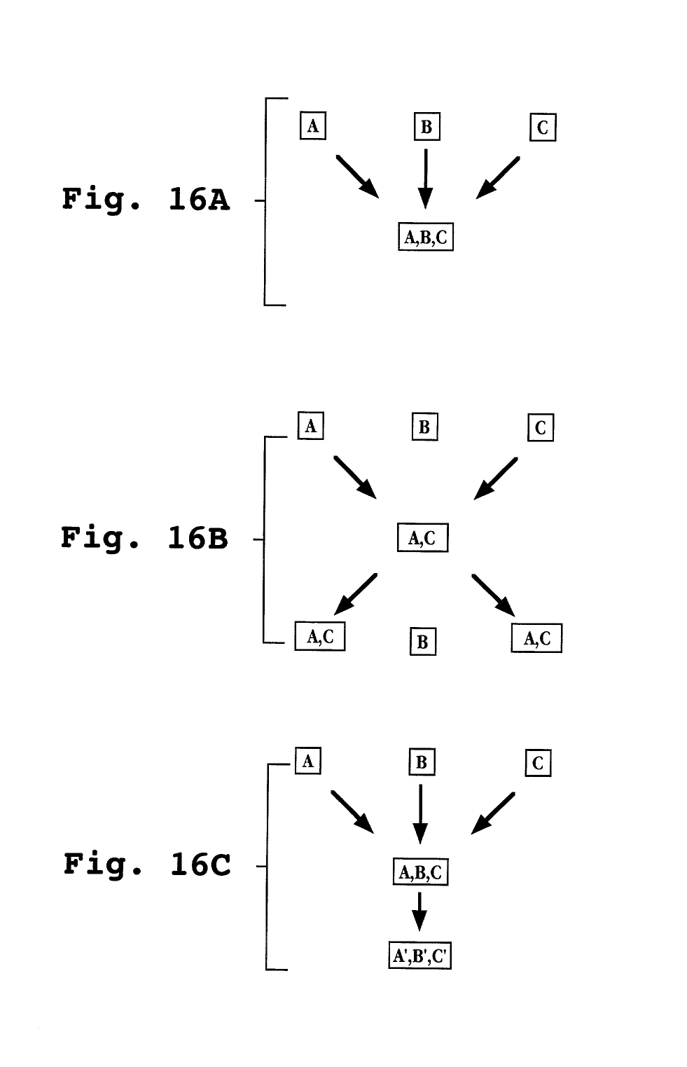

Loading of the reservoirs and reaction channels is as in the embodiment described above in subsection A. FIGS. 16A-16C illustrate some of the sample or sample-component manipulations that are possible in the present invention. In FIG. 16A, the passive-flow device of FIG. 15A is operated to move components A, B, and C from each of three adjacent reaction regions into a common hold reservoir, for purposes of combining and mixing the components. In FIG. 16B, components A and C from different selected regions are transferred to the common hold reservoir, where they are mixed, then redistributed to the original reaction regions, or transferred to a second multi-site reaction channel. This operation would require active control over the flow through each region, and therefore would reflect an operation on the device of either FIGS. 15B or 15C. FIG. 16C illustrates a first operation to combine components A, B, and C from different reservoirs, and a second operation to convert the mixed components to new products A′, B′, C′.

An embodiment having a common supply reservoir and region-specific hold reservoirs would be constructed, and would function similarly, as can be appreciated. In particular, the side channels connecting a common supply reservoir to the individual reaction regions are designed to have equal flow resistance and/or a valved to allow selection of specific reaction regions for input from the common supply reservoir.

C. Device with Common Supply and Hold Reservoirs

In another general embodiment, both the supply and hold reservoirs servicing the multiple reaction regions in a channel are common reservoirs, each connected to the reactions regions through a suitable side-channel manifold of the type discussed above. As in subsection B, this configuration may additionally include region-specific reservoirs. For example, a reaction region may have a common first hold reservoir for moving material into and through the reaction region into a common reservoir, and a region-specific second hold reservoir, for use in sampling a portion of the reaction material contained in each region.

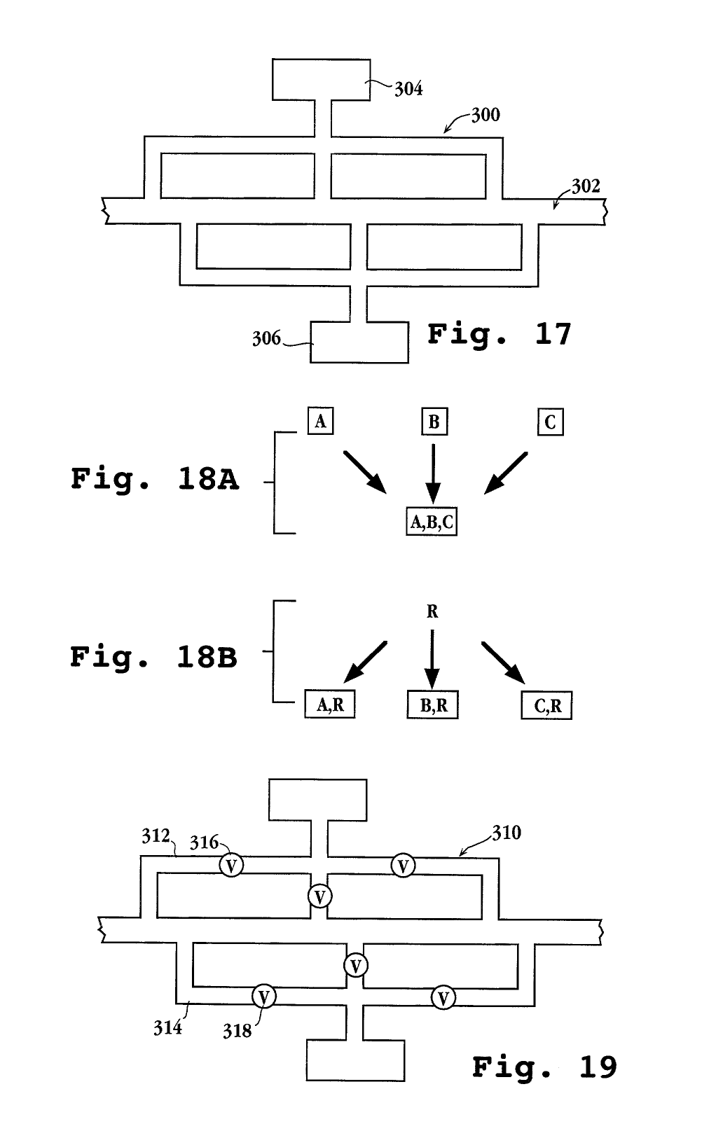

One embodiment having both supply and hold common reservoirs is illustrated at 300 in FIG. 17. The embodiment is designed for passive transfer of a common reagent simultaneously from the common supply reservoir to the plural reaction regions, as shown in FIG. 18B, or simultaneous transfer of material from the plural reaction regions to a common hold reservoir, as shown in FIG. 18A. The device includes a reaction channel 302 with multiple reaction sites, a common supply reservoir 304 and a common supply reservoir 306. As above, the side channels connecting each common reservoir to the individual reaction regions are designed to have substantially equal flow resistances, either by virtue of having common lengths, variable cross-sectional areas, or some combination of the two.

A device with a similar reservoir configuration, but with flow-control elements in each side channel, is illustrated at 310 in FIG. 19. Here each side channel connecting a common reservoir to an individual reaction region, such as side channels 312, 314, is provided with a control valve, such as valves 316, 318, respectively, for controlling fluid flow through that channel. This embodiment allows various common reagent supply and selection-region removal and mixing operations, as can be appreciated.

From the foregoing, it can be appreciated how various objects and advantages are achieved with this aspect of the invention. The service reservoirs, e.g., supply and hold reservoirs, allow (i) introduction of region-specific reagents into all or selected reaction regions, to initiate the simultaneous region-specific reactions, (ii) sampling of reaction material in each or selected channels, during or after reaction, (iii) removal of reaction material from selected regions, (iv) mixing of reaction material from selected regions, and (v) further reaction or processing of reaction material.

In particular, the device and method allows for sampling of reaction components or kinetics during or after simultaneous reactions in a multisite reaction device, and further processing operations, e.g., extraction, removal, mixing, reacting, or separating reaction components based on the results of the sampling. This general feature is expanded upon in the next aspect of the invention.

V. Apparatus with Multisite Reaction Channel and Sample-Processing Stations

In another aspect, the invention includes a sample-handling system for carrying out multiple reaction and processing steps, including simultaneous multisite reaction steps of the types discussed above. The system or apparatus includes a microchannel device having a reaction channel of the types described above, for carrying out simultaneous multiplexed reactions on a bulk-phase sample, and additionally includes one or more sample-preparation stations upstream of the reaction channel for carrying out one or more selected sample-preparation steps effective to convert a sample to such bulk-phase medium, and/or one or more product-processing stations downstream of the reaction channel, for processing products generated in one or more of said reaction regions.

The system also includes structure for transferring solvent or solvent components between one of the sample-preparation stations and one or more selected reaction regions in the reaction channel, and between one or more selected reaction regions in the reaction channel and one of the product-processing stations. The transfer structure, which can include electrodes for electrokinetic movement, pressure gradients, and other transfer means of the types discussed above, is under the control of a control unit which coordinates movement of sample, reagent, and reaction-product material in and out of selected stations.

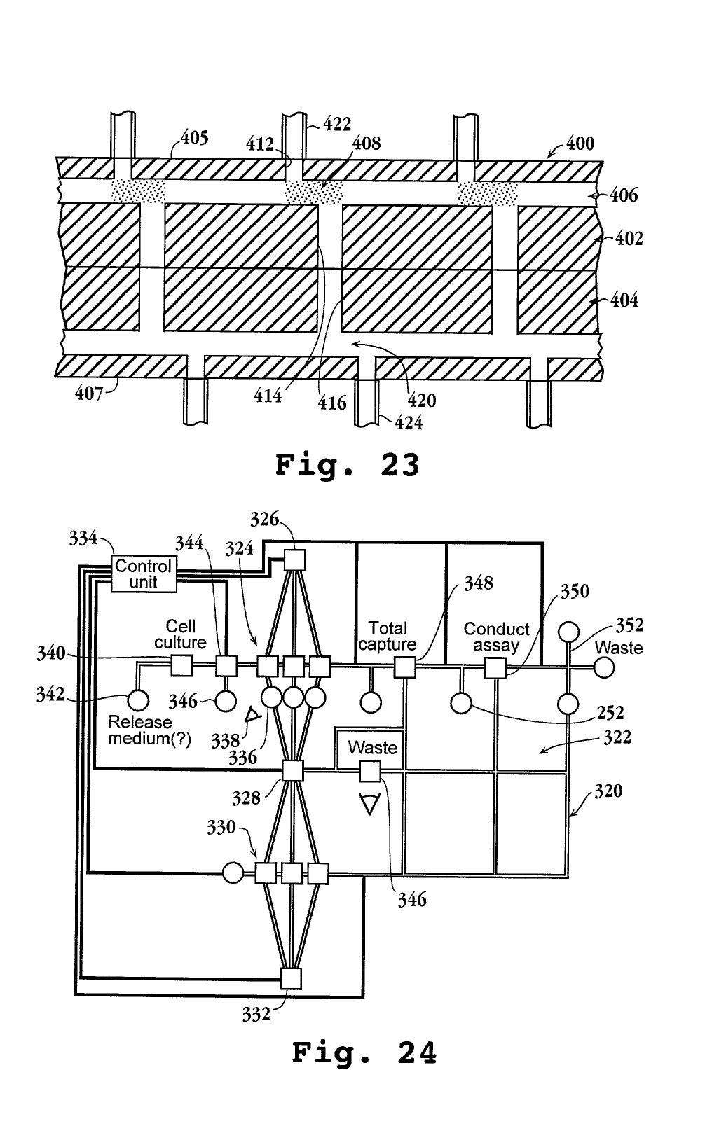

FIG. 23 is a schematic view of a system or apparatus 320 constructed according to the invention. The system includes a microchannel 322 device of the type described above, formed on a substrate and having a multisite reaction channel formed therein, but containing additional sample-processing and reagent-supply stations or reservoirs for carrying out a variety of desired operations in the device. The device is formed, as above, by microfabricating a substrate with a desired configuration of microchannels, reservoirs, reaction chambers, connecting channels, and control elements (which form part of the transfer structure), such as valves or electrodes, according to methods and processes discussed above, and well-known in the art.

The device illustrated in FIG. 23 includes a first multisite reaction channel 324 of the type described above also having a common supply reservoir 326, a common hold reservoir, and control elements (not shown) in the connecting side channels for controlling transfer of material in and out of the reaction regions in the channel. Reservoir 328, in turn, functions as the supply reservoir of a second reaction channel 330 which is also provided with a common hold reservoir 332. All of the transfer and control elements are under the control of control unit 334, as indicated, for producing selected reagent input to the first-channel regions, movement of selected reaction components into reservoir 328, and further mixing and/or processing of the reaction components, e.g., by transfer the components to the second reaction chamber, or to selected regions therein, via reservoir 328.

Also shown in the figure are sampling stations, such as station 336 contained in line with side channel servicing the reaction regions in channel 324. Material in the sample station can be assayed or quantitated by a detector 338, e.g., a biosenser or optical detection system, for determining the amount and/or presence of given components in the station. Based on the results of the assay, the components may be moved through reservoir 328 into one or more reaction regions in channel 330, or transferred to other processing or waste stations in the device. Considering the upstream functions in the device, these generally include a culture station 340 at which cells may be cultured, and an upstream release medium used for supplying selected reagents and/or liquid to the culture station. Alternatively, the original sample material is a non-cell sample, e.g., homogenized tissue or the like, the cell culture station can be replaced by a tissue processing station where the tissue is, for example, treated with enzymes or detergents.

Upstream of the cell culture station is a reservoir 342 for holding culture medium, medium supplement, and/or release medium for releasing cells in station 340, rather than by a lysis buffer. Downstream of station 340 is a lysis or treatment station 344 at which cells or other particulate sample material can be lysed, by supply of a lysing agent from a reservoir 346 into this station. Suitable detergent and/or enzymatic lysing buffers are well known.

Although not shown, the upstream stations may also include a filter or other capture station for capturing unwanted material, e.g., cell or tissue particulate matter, as it is transferred from the lysing station to the first multisite reaction chamber.

Material supplied to the first reaction channel is referred to herein a bulk-phase material, and typically includes cell or tissue lysate, or purified or partially purified nucleic acid fractions. The material is introduced into the channel to fill each of the reaction regions in the channel. The bulk-phase material is now contacted with region-specific reagents, as described above, to carry out simultaneous multiplexed reactions on the bilk phase sample material.

Following this reaction, the reacted components may be selectively removed, mixed, and/or sampled, before being sent to a downstream processing station. As noted above, one downstream processing station is a second multisite reaction chamber, where one or more isolated or mixed components from the first channel can be further reacted with each of a plurality of different reagents. For example, the first reaction channel may contain sequence-specific PCR reagents, for amplifying each of a plurality of different nucleic targets in a sample, and the second reaction channel may contain sequence-specific reagents for use in detecting specific sequences, e.g., labeled ligase- or exonuclease-dependent probes, based on the binding of the probes to the nucleic acid material amplified in the first channel.

Alternatively, or in addition, individual or combined reaction products from the first channel can be transferred to a waste reservoir 346 or to other downstream processing stations, such as a capture station 348, where some or all components are captured, and/or concentrated, and from here to an assay station 250, past a reagent reservoir from which assay reagents can be supplied to the sample components. In the assay station, further reactions may take place, or the reacted material may simply be assayed. Thus, the assay station may include a biosensor of a type suitable for fabrication on a microchannel device, or an external or internal optical detection system.

Alternatively, or in addition, sample material may proceed to a separation station, such as a microchannel electrophoresis device 352 for sample-component separation and detection. As will be appreciated from the drawings, the just-described downstream processing functions are accessible both to individual or mixed components from the first or second multisite reaction channel.

FIG. 20 illustrates one type of coupling between first and second reaction channels in the apparatus of the invention. The two channels are indicated at 354 and 356, each having a plurality of discrete reaction regions, such as region 358 in channel 354 and region 365 in channel 356. Each region in the first channel is serviced by a separate supply reservoir, such as reservoir 360, and is serviced at its “output” side, by a side channel, such as channel 362, directly connecting a downstream end of one region with an upstream end of a corresponding region in the second channel. Each such side channel has a control element, such as reservoir 366 in side channel 362, which can be used to control liquid movement through the channel, as described above with reference to FIG. 15B. In the figure, the voltages applied are such as to transfer the reaction material in region 358 in the first channel to the corresponding region 365 in the second channel.

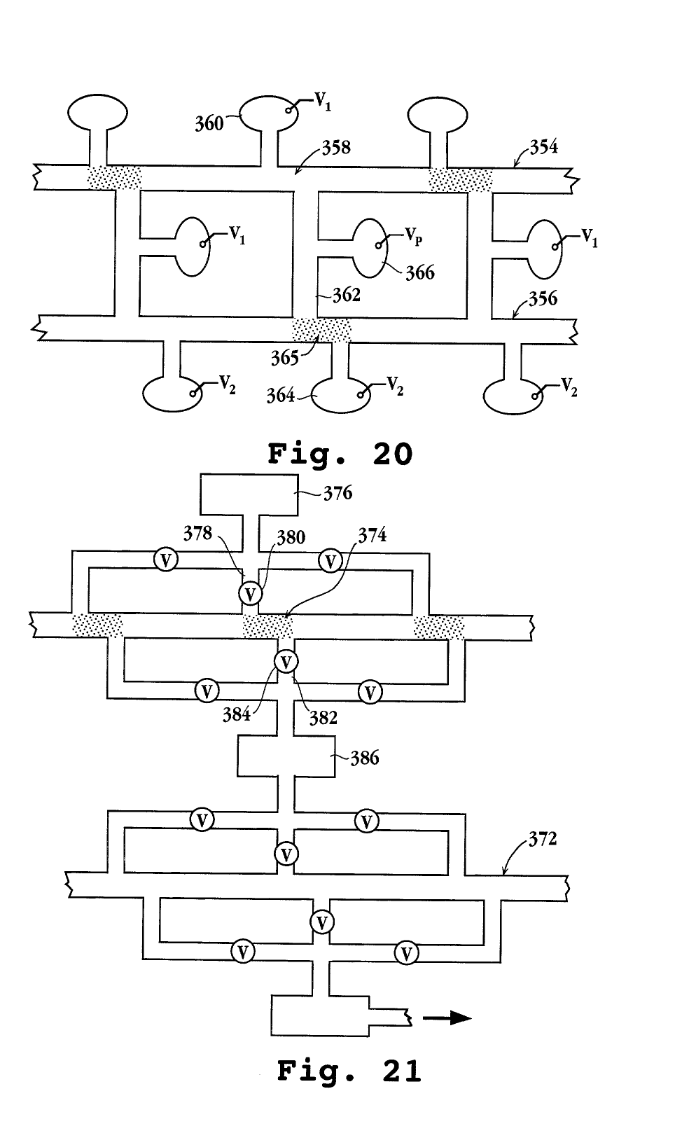

A more versatile channel-to-channel configuration is illustrated in FIG. 21, in which first and second multisite reaction channels 370, 372, respectively, are both supplied and connected by common reservoirs which can be controlled for selected-region transfer. In particular, channel 370 has a plurality of regions, such as region 374, which are connected at their upstream ends to a common supply reservoir 376 through associated side channels, such as channel 378, each of which is provided with a control element, such as valve 380, as detailed above. The same regions are connected, at their downstream ends, to a common hold reservoir 380 through associated side channels, such as channel 382, each of which is also provided with a control element, such as valve 384. The common-reservoir/controlled side channel configuration is like that described with reference to FIG. 19 above, providing the ability to transfer material into and out of each region separately or in parallel, and to mix and combine any combination of reaction products in the hold reservoir.

In the present embodiment, the common hold reservoir serves as the supply reservoir of the second reaction channel, as can be seen, allowing individual or combined components to be distributed selectively, or in parallel to the individual reaction regions in channel 372, where the reaction products from the first channel can undergo further region-specific reactions. Following the second reaction, the products in the second-channel regions can be further removed, combined, or simply transferred downstream within the channel.

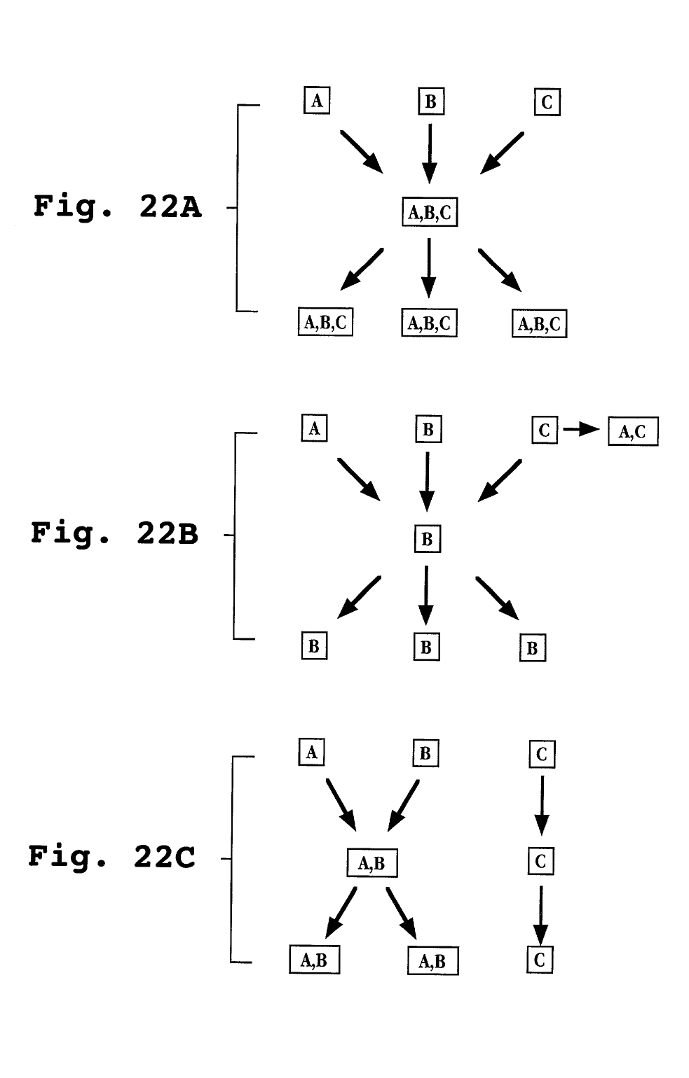

FIGS. 22A-22C illustrate the type of combined reactions that can be carried out with the above coupled-channel systems. In the operation illustrated in FIG. 22A. reaction components A, B, and C from the first channel are combined in a common hold reservoir, then redistributed as combined components to the each of the reaction regions in a second channel.

A similar operation is illustrated in FIG. 22B, except that component B is selectively moved to the common (or individual) hold reservoir, and from this chamber to each of the reaction regions in the second channel. Components A and C remaining in the first channel are then transferred to another station in the device.

As another permutation, in FIG. 22C, components A and B from the first channel are combined and redistributed to two reaction regions of the second channel, while individual component A is transferred directly to an individual region in the second channel. Various other permutations of these transfer operations are possible.

FIG. 23 shows another embodiment of the device, indicated at 400, in which the two multisite reaction channels are formed in separate substrate layers, such as layers 402 and 404. The layers may be bonded together, to produce a unitary device, or may be removably attached, as described below, to allow different device reaction functions to be substituted with others during the course of the operation of the device. For purposes of discussion, the layers will be referred to as upper and lower layers, and the exposed layer surfaces, as upper and lower surfaces, indicated at 405 and 407, respectively. Each layer, in turn, in typically formed of a base substrate, in which channel and reservoir features are formed, and a cover bonded to the substrate for enclosing the channels and providing ports or opening through which material can be supplied to the channels, as is conventional in microchannel device construction.

As seen, layer 402 has formed therein a channel 406 containing a plurality of reaction regions, such as region 408, and, for each region, a side supply channel, such as channel 412 communicating with the upstream end of each region. The downstream end of each region is similarly serviced by an outlet side channel, such as channel 414, which mates with an associated supply channel, such as channel 416, communicating with the upstream end of a corresponding reaction region 420 in the channel formed in layer 404.

In the present invention, reservoirs for adding material to, or removing material from the individual reaction regions is provided capillary tubes, such as tubes 422, 424 which are removably insertable into ports formed in the upper and lower layers, respectively, and accessible from the device's upper and lower exposed surfaces. Operation of the device is as described above, where the two-layer device may additional include transport structure for moving material selectively from one ore more reaction regions in the upper channel to those in the lower channel.

In one embodiment, the two layers forming the device are removably attached to one another, and interchangeable with other layer modules, for performing various types of sample processing in one module, then, based on the type of further processing needed, mating that module with one of a plurality of different modules, or identical modules with different and/or fresh reagents, to continue to process one more components produced in one module in a successive module. The modules are, of course, designed with alignment pins or the like to ensure proper alignment of mating channels, such as channels 414, 416, when the modules are placed together. The same concept may can also be implemented in a side-by-side module configuration, where two or more modules are placed operatively together along sides having aligned channels for transfer of material therebetween.

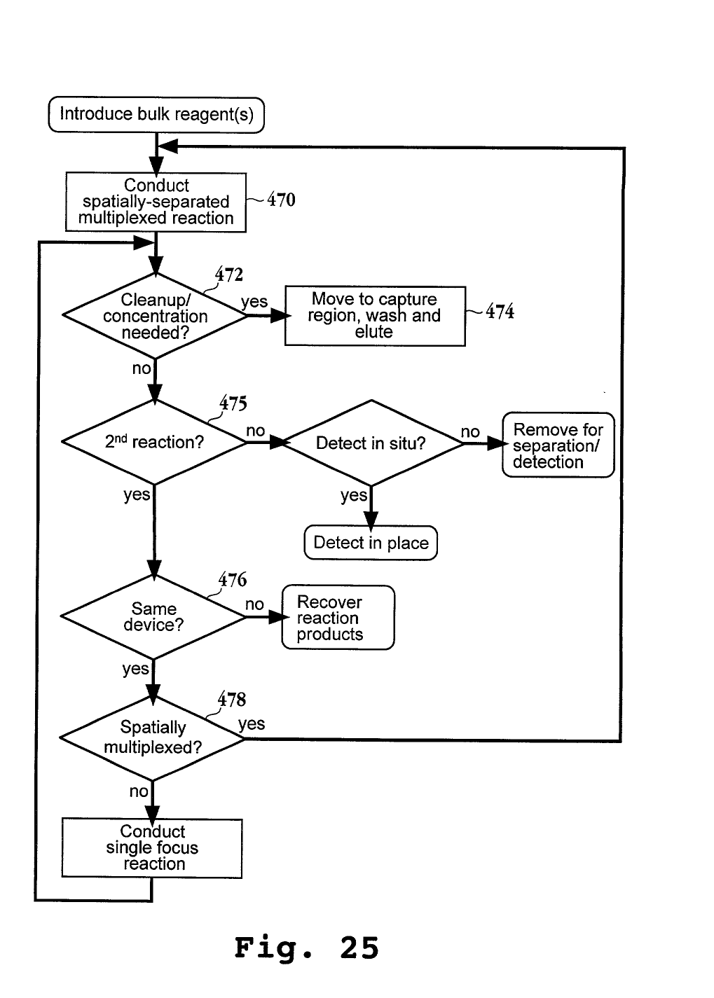

FIG. 25 illustrates a typical multi-step operation that can be carried out by the apparatus of the invention, preferably under the control of the apparatus control unit. That is, the control unit is preprogrammed to carry out the following operations, partly in respond to information provided during the operation.

Initially, bulk-phase sample and reagents are prepared, as above, and supplied to the multisite reaction channel, as at 470. Following the reaction, the material may be transferred to a downstream station for cleanup or concentration, as at 472, and from here, to capture and elute, e.g., to remove unwanted components, or to collect desired samples, as at 474. If, based on an interrogation of the sample, or as part of a preprogram, it is necessary to conduct a second reaction, as at decision point 475, the material may be recycled through the original reaction channel, or transferred to a second multisite reaction channel, as at 476, where an additional multiplexed reaction is carried out. From here the products may be recovered, e.g., for separation and/or assay, or returned to one of the upstream stations in the device, as at 478. Alternatively, after either the second reaction or the second multiplexed reaction, the material or selected-region components can be detected and or removed for separation.

It will be appreciated that a variety of other reaction protocols involving sample preparation, reaction, and further processing can be executed using a suitably programmed control unit. In addition, at any stage of the operation, new reagents or reaction products can be added to or removed from the device, e.g., by capillaries inserted into exposed ports.

The following examples are offered by way of illustration and not by way of limitation.

### EXAMPLE 1

### Preparation of a Channel Region with Primers and Performing PCR.

A. Synthesis of SPDP-BSA-benzophenone

First, mix 10 mg each of succinimidyl-3-(2-pyridylthiopropionate) (SPDP) and 4-benzoylbenzoic acid in a brown bottle and dissolve the mixture in 1 mL anhydrous dimethylformamide. Next, dissolve 100 mg bovine serum albumin (BSA) in 6 mL phosphate buffered saline, pH 7.2. Combine the two solutions by adding ˜50 uL of the DMF solution to the solution of BSA, with vortexing, every 30 minutes. Keep the reaction solution in the dark, agitate the reaction solution on a shaker (150 rpm) between additions. After the additions are complete continue shaking the solution at room temperature until 2 days have elapsed. Then, dialyze the reaction against water for 1 day, in the dark, with three changes of water. Centrifuge the solution for 10 min at 3000 rpm, and collect the supernatant. Lyophilize the supernatant to dryness, and store the product, SPDP-BSA-benzophenone, as a solid at −20C. For use in experiments, prepare a 10 mg/mL solution of SPDP-BSA-benzophenone in 1× PBS buffer (pH 7.2).

B. Surface Attachment of SPDP-BSA-benzophenone in a Channel and Formation of Devices with Region-Specific Capture Nucleic Acids.

A polycarbonate substrate with channels 50 um deep, 120 um wide and 50 mm long was prepared by compression molding. The surface of the plastic substrate was washed with water, dried with a tissue, and ˜30 uL of the 10 mg/mL solution of SPDP-BSA-benzophenone was pipetted into the channel. A rubber gasket was placed on the surface of the substrate surrounding the channel, and on top of the gasket was placed a mask prepared with black electrical tape and a glass slide. A portion of the tape was cut out to provide irradiation to a 3 mm long section of the channel. Another slide was positioned under the substrate for support, and the 4-layer assembly (mask, gasket, substrate, support slide) was clamped tightly. The assembly was exposed for 20 min to a collimated beam of light from a 100 W mercury arc lamp. After disassembly the substrate was washed three times each with 0.05% Triton X-100 and water. The channel was thus prepared with a region carrying an activated disulfide bond-forming group, where the region was defined through masked photodeposition of the light-sensitive reagent. A capture nucleic acid, 1, having a terminal thiol group was prepared, and 250 pmol were dissolved in 50 uL of 0.5 M carbonate buffer. A portion of the solution was pipetted into the channel at the irradiated region, and incubated at room temperature for 2 hr. The substrate was then washed with water, dried, and the open channels of the substrate were sealed by thermal lamination with a 40 um thick film of PMMA (MT-40).

C. PCR

PCR primers 2 and 3, targeting the beta-actin gene, were prepared with a target specific 3′ end portion, a 5′ end portion complementary to the sequence of the capture nucleic acid 1, and a non-amplifiable polyoxyethylene spacer moiety linking the two portions. The primers were combined in a PCR reaction mix consisting of 1× PCR buffer II, 200 uM TTP, 200 uM dCTP, 200 uM dGTP, 40 uM dATP, 160 uM F-dATP (fluoresceinated dATP), 1.5 mM MgCl₂, 0.01% BSA, 0.5 uM primers 2 and 3, and optionally a diluted sample of an unlabeled product solution of the beta-actin amplicon as template. The PCR mix was added to the channels prepared as above. Samples with and without the template were prepared. The reservoirs were taped closed, and the substrates were placed on an MJ Research thermocycler unit with a flat block and thermocycled according to the protocol: denature at 92 C. for 2 min; 26 cycles of 92 C. for 1 min, 54C for I min, and 72 C. for 30 sec; final extension at 72 C. for 5 min, and hold at 4 C. until retrieved. The reaction was also performed in a standard PCR tube as a control.

After the reaction was complete the solution was removed from the channels and tubes and analyzed by polyacrylamide gel electrophoresis. Also, the channels were refilled with TENSS buffer (100 mM Tris, 25 mM EDTA, 300 mM NaCl, 0.1% dextran, 0.01% salmon sperm DNA) and the channels examined by fluorescence microscopy. PAGE analysis revealed the presence and absence of product amplicon bands where the reaction was carried out with and without template, respectively. The image analysis showed that the irradiated region of the channel treated with SPDP-BSA-benzophenone gave a strong fluorescent signal after thermocycling the reaction mix containing the template whereas the non-irradiated regions yielded no signal. No fluorescence was observed in the treated channels when the template was not in the reaction mix. Such results indicate that the reaction produced amplicons with fluorescent labels incorporated in the strand, and the amplicons, generated with single-stranded ends because of the non-replicable moiety, hybridized to the capture nucleic acids immobilized on the surface of the channel.

### EXAMPLE 2

### Preparation of a Channel Region with Capture Nucleic Acids and Measurement of the Binding Capacity.

A. Synthesis of biotin-BSA-benzophenone

The procedure for preparing biotin-BSA-benzophenone was the same as that given above for the preparation of SPDP-BSA-benzophenone, replacing SPDP with (biotinylamidocaproylamido)caproic acid N-hydroxysuccinimide (Biotin-X-X-NHS).

B. Preparation of Streptavidin-Coated Channels

First, biotin-BSA-benzophenone was attached to channel surfaces by the same methods as described above for SPDP-BSA-benzophenone. After irradiation and washing away unbound materials, a 0.1% solution of streptavidin in TE buffer, pH 8.0 was added to the channel and incubated at room temperature for 30 min. The channel was then washed three times each with 0.05% Triton X-100 and water. The substrate was then dried, and the open channels of the substrate were sealed by thermal lamination with a 40 um thick film of PMMA (MT-40).

C. Demonstration of the Formation of Reaction-Specific Reagent Regions

An oligonucleotide duplex was prepared using one biotinylated oligo, 4, and one fluorescein-labeled oligo, 5. Equimolar solutions of 4 and 5 were combined in TENSS buffer with a final concentration of 10 uM. To ensure formation of the duplex, the solution was heated to 70 C. for 15 min and then left to cool at room temperature for 30 min prior to use. This stock solution was further diluted to 1 uM concentration and introduced into the treated channel. After 10 min incubation, the solution was removed and the channel rinsed with 0.5 mM MgCl₂, 50 mM Tris [pH 8.0] buffer. Imaging the channel by fluorescence microscopy revealed a fluorescent signal in the region of the channel that was irradiated through the mask. Irradiation effected the deposition of biotin-BSA-benzophenone, which in turn bound the streptavidin to this region. The oligo duplex binded to this region via complex formation between the biotinylated oligo and the surface streptavidin, which yielded the signal due to the labeled oligo hybridized to the biotinylated oligo.

To confirm the nature of this localization of the duplex, a competitor oligo, 6, was added at a concentration of 50 uM in 0.5 mM MgCl₂, 50 mM Tris [pH 8.0] buffer to the channel. The fluorescent signal disappeared within minutes. The sequence of 6 was designed as a competitor to oligo 5, having a longer region that is complementary to the capture oligo 4 and thus able to cause the displacement of oligo 5.

D. Measuring the Surface Binding Capacity

The surface binding capacity of a surface treatment for the carrying of reaction-specific reagents determines the solution concentration of these reagents when released for the performing of a reaction, or the surface concentration of a heterogeneous reagent employed in immobilized form. The surface binding capacity of channels treated with biotin-BSA-benzophenone and streptavidin was determined by two methods. In one, duplexes of 4 and 5 were preformed, bound to the surface, and the amount of fluorescent signal released from the channel upon addition of the competitor 6 was quantified. In the second, the capture oligo 4 was first bound in the channel to create a channel surface carrying one member of a specific binding pair. Then the reagent 5 was added to the channel, where the two oligo binding pair members formed the duplex. Again, the competitor was added to cause the release of the labeled oligo, which was collected and quantified. The released solutions were brought to the same volume using the buffer solution, and a series of solutions of known concentration of the labeled oligo were used to prepare a standard curve relating fluorescence intensity to the amount (or concentration) of fluorophore. The results indicated that the same binding capacities were obtained by either method of preparing the channel. The binding capacity varied with the concentration of 5 introduced into the channel, increasing to a binding capacity of about 0.08 pmol/mm² as the concentration of 5 reached 1.5 uM.

### EXAMPLE 3

### Fabrication of Regions of Reaction-Specific Reagents.

As in Example 2C, reaction-specific reagent regions were prepared. In Example 2C, the first layer in the structure was defined by the irradiation pattern and subsequent layers conformed to this spatial definition, whereas, in this experiment the underlying layers were prepared uniformly along the channel and the localized reagent regions were defined by the localized delivery of reagents to the treated surface. Channels were prepared in polycarbonate substrates by either milling or compression molding. The channels were washed with soap and rinsed with MilliQ water. A 1% solution of biotin-BSA-benzophenone was pipetted into the channels and irradiated for 15 min with a 100W mercury arc lamp through a glass slide filter. The channels were then rinsed three times each with 0.05% Triton X-100 and deionized water and then dried. The channels were then treated with a 0.1% solution of streptavidin. After incubating at room temperature for 30 min, the channels were rinsed three times with 1X PBS solution, or alternatively a 1× PBS, 1% BSA solution. Another duplex of one biotinylated oligo, 7, and one fluorescein-labeled oligo, 8, was prepared in TENSS buffer to a final concentration of 1 uM, annealed as described above and spotted in the channels to define regions of various lengths and number in a series of channels. The channels were rinsed of the excess, unbound materials by washing three times with 0.5 mM MgCl₂, 50 mM Tris [pH 8.0] buffer. The washes were collected and measured for fluorescence. The results demonstrate that by the final wash no more fluorscent signal is being recovered from the channel. The fluorescein labeled oligo was then stripped off the channel surfaces by adding a denaturing release solution of 70% aqueous formamide. A standard curve relating fluorescence intensity to amount of labeled oligo was prepared using a series of dilutions of known concentration of the labeled oligo. Control experiments demonstrated that the release solution does not cause release of the capture oligo 7. The series of regions created and quantified are summarized in the table below.

| | | | | | | | |
|---|---|---|---|---|---|---|---|
| No. of regions | 1 | 5 | 4 | 3 | 2 | 2 | 2 |
| Length of region | 18 | 2 | 2 | 2 | 2 | 3 | 4 |
| Total effective length | 18 | 10 | 8 | 6 | 4 | 6 | 8 |
| Signal of released | 3.29 | 1.47 | 0.85 | 0.63 | 0.42 | 0.94 | 0.88 |
| oligo | | | | | | | |

The relationship thus determined between the surface capacity and the size and number of regions carrying nucleic acid reagents demonstrates the ability to fabricate such regions having a useful amount of reagent for reactions.

### EXAMPLE 4

### Multiplex PCR Using Devices of the Subject Invention with Primers Releasably Bound via Hybridization

Channels were prepared in polycarbonate substrates of dimension 0.4×0.8×18 mm. Ports were made by drilling holes at the channel ends to the opposite surface, and the channel was enclosed by laminating a thin polycarbonate film to the side of the substrate with the open channels. The surface of the channel was treated as described in Example 3 with biotin-BSA-benzophenone and streptavidin. Then, primer sets were introduced into separate regions by incubating solutions of primer/capture nucleic acid duplexes in distinct portions of the channels. A 3-plex reaction using three the primer pairs was performed in a channel device with each primer set localized to separate regions, and the three combinations of 2-plex reactions were also performed with each primer set localized to separate regions. For use in the device, each primer of a pair was prepared with the same 20-mer capture sequence extending from the 5′ end of the primer sequence, and a capture probe was prepared with the complementary capture sequence and a 3′ biotin. Thus, capture nucleic acid 9 for primers 10 and 11; capture nucleic acid 12 for primers 13 and 14; and capture nucleic acid 7 for primers 15 and 16. Each set was prepared in duplex form using a molar ratio of 2:1:1 of capture nucleic acid:primer:primer, in TENSS buffer, annealed as described above. Each set was introduced separately into the channel and incubated for 30 min. To prepare the 2-plex reactions in localized regions each primer set was introduced via each of the two terminal ports with the solutions only filling around half the channel so as not to permit mixing. For the localized 3-plex reaction, two sets were introduced again via the two terminal ports to only one-third the channel length. After these binding reactions, the third set set was introduced to the middle region for binding to the remaining free sites in that region. After the primer sets incubations the channels were rinsed with 1× PBS. The homogenous 3-plex reaction was also performed in tubes and in the channels. In the case of the channels the primers were supplied with the PCR reaction mix, but the surfaces were treated with biotin-BSA-benzophenone and streptavidin, though no capture nucleic acids were added.

After loading the channels with the bound primer reagents, the channels were filled with a standard PCR mix. The loaded plastic device was sealed with pressure sensitive adhesive (PSA) film, placed on a PE 9700 thermocycler instrumet (the channels were designed to fall on top of the metal surface and not on top of the holes for holding tubes) with a plastic shim on top of the device, and secured in place by closing the lid. The shim acted to transfer the pressure of the lid down to the PSA film. The thermocycler was programmed as follows: 94 C., 10 min; 35 cycles of 94° C., 45 sec; 58° C., 30 sec,; 70 C., 45 sec; with a final extension at 70 C. for 10 min. Following the reaction the solutions were removed from the channels and analyzed by 2% agarose gel electrophoresis.

This homogenous 3-plex reaction failed to produce one of the three amplicons in significant amounts in all the samples run. However, with the primers localized to separate regions in the channel, in both the cases of 2-plex and 3-plex reactions, the reactions proceeded to yield all the expected amplicon products in the expected amounts as determined by gel analysis.

The results of this experiment demonstrate that within one fluidly connected channel, localized primer sets can react in combination with the same common reagents provided in the bulk. Furthermore, as observed with the 3-plex reaction, starting with spatially separated reaction-specific reagents, wherein the reactions proceed in substantial isolation may provide a better yield of the different products than when performed as a typical multiplex wherein the reagents are fully mixed.

### EXAMPLE 5

### Multiplex PCR Using Devices of the Subject Invention with Primers Releasably Bound Via Ligand Binding and a Demonstration of the Substantial Isolation of Reaction Regions

Devices as described in Example 4 were again fabricated and prepared with biotin-BSA-benzophenone and streptavidin treated surfaces. Two primer pairs, 17, 18 and 19, 20, were prepared with biotinylated 5′ ends. Solutions of various combinations of the primers were prepared in 1× TE buffer, and introduced into the channel and incubated for 30 min at room temperature to establish channel surfaces with primers bound via the biotin/streptavidin linkages. The primer combination prepared were as follows: set A (17,18,19,20); set B (17,18); set C (19,20); set D (17,19); set E (18,20). Sets A, B and C are proper combinations of primers in that they yield amplified products, with set A being a 2-plex reaction which was established to consistently produce both amplicons well. Sets D and E however are improper combinations that in isolation do not yield any amplified products. Channels were prepared in duplicate in the following manner: 1: set A; 2: sets B and C in separate regions of the channel with no gap between the regions; 3: sets B and C in separate regions of the channel with a 1 mm gap between regions; 4: sets D and E in separate regions with no gap between the regions; and 5: sets D and E in separate regions with a 1 mm gap between regions. After incubating the primer solutions in the channels the channels were again rinsed with 1X PBS. As in example 4, the PCR reaction was introduced into the channels, the ports were sealed with PSA film, the device and shim secured in the thermocycler, and the reaction performed using the same cycling protocol listed above. The solutions were removed after cycling, combined with a loading buffer and analyzed by 2% agarose gel electrophoresis.

The 2-plex reaction of 1 produced the expected two bands of the two amplicons, and the reactions of 2 and 3, with the two different primer pairs in separate regions also produced the same two bands in similar yields. Reaction 4 produced significantly less product of each of the two amplicons, with bands discernible by eye but too feint to accurately quantify. Reaction 5 failed to produce any visible bands. Each reaction was performed in duplicate, and gave identical results.

The results of this experiment demonstrate the utility of using directed binding of ligand-labeled (biotinylated) primers to receptor-bearing (streptavidin) surfaces for establishing defined regions of different reaction-specific reagents. This and other experiments have also demonstrated that primers bound via ligand/receptor complexes are released from the surface into solution in the course of the thermal cycling protocol by thermal denaturation of the complex. Also, by setting up wrong primer combinations of a functional 2-plex set, this experiment also demonstrates that convective mixing is not occuring on the time-scale of the reaction and thus the reactions are regionalized according to the placement of the reagents and proceed in substantial isolation.

### EXAMPLE 6

### Amplification Using Secondary Primers

Devices as described in Example 4 were again fabricated and prepared with biotin-BSA-benzophenone and streptavidin-treated surfaces. Capture nucleic acid/primer pair, (probe 7/primers 15, 16 and probe 12/primers 13, 14) solutions were separately prepared in TENSS solution as described above, and separately introduced into a series of channels, and incubated for 30 min. After rinsing the channels of the unbound probes, a standard PCR reaction mix was added, with varying amounts, concentrations of 0, 0.1, 0.3 and 0.5 uM, of a corresponding secondary primer (primer 21 and 22, respectively) in the mix. The secondary primers have the same sequence as the capture sequence portion of the primers. The devices were sealed and thermocycled as previously described. The reaction products were analyzed by agarose gel electrophoresis.

The reactions each produced the expected amplicon product. The amount of product however increased with increasing concentration of the secondary primer, ultimately yielding approximately 100% more product when present at 0.5 uM concentration as determined by the band intensities for both primer sets. Separate control experiments lacking the primary primers, 15 and 16, or 13 and 14, failed to produce any products.

This experiment demonstrates the utility of secondary primers for boosting the PCR amplification yield.

List of Sequences
SEQ ID 1
5′ thiol AAC AGC TAT GAC CAT GCG CCA GGG TTT TCC CAG TCA
CGA C 3′
NOTE: thiol = thiol modifier C6
Sequence Type: probe
SEQ ID 2
5′ F CCT GGC GCA TGG TCA TAG CT P TCA CCC ACA CTG TGC
CCA TCT ACG A
NOTE: F = 6-(fluorescein-5(6)-carboxamido)hexyl; P = hexaethyleneglycyl
Sequence Type: primer
SEQ ID 3
5′ F CCT GGC GCA TGG TCA TAG CT P CGG AAC CGC TCA TTG CC
NOTE: F = 6-(fluorescein-5(6)-carboxamido)hexyl; P = hexaethyleneglycyl
Sequence Type: primer
SEQ ID 4
5′ B AAC AGC TAT GAC CAT GCG CCA GGG TTT TCC CAG TCA CGA
C
Sequence Type: probe
SEQ ID 5
5′ F CCT GGC GCA TGG TCA TAG CT
NOTE: F = 6-(fluorescein-5(6)-carboxamido)hexyl
Sequence Type: primer
SEQ ID 6
5′ GTC GTG ACT GGG AAA ACC CTG GCG CAT GGT CAT AGC TGT T
Sequence Type: probe
SEQ ID 7
5′ ACA TCG GAC GCA GTG GAC CTC ACG TCT ACA AGT CGC CTG
APB
NOTE: B = biotinTEG; P = triethyleneglycyl
Sequence Type: probe
SEQ ID 8
5′ AGG TCC ACT GCG TCC GAT GTP F
NOTE: 6-(fluorescein-5(6)-carboxamido)hexyl; P = hexaethyleneglycyl
Sequence Type: primer
SEQ ID 9
5′ CTG ATG CCG AGA GCT GCC AAG CCC ATA TAC GAT GCC TCG
APB
NOTE: B = biotinTEG; P = triethyleneglycyl
Sequence Type: probe
SEQ ID 10
5′ TTG GCA GCT CTC GGC ATC AGT CAT CCA TCA TCT TCG GCA
GAT TAA
Sequence Type: primer
SEQ ID 11
5′ TTG GCA GCT CTC GGC ATC AGC AGG CGG TAG AGT ATG CCA
AAT GAA AAT CA
Sequence Type: primer
SEQ ID 12
5′ GCT ATG CGA CCG ACC TAC CGT TTG AGC CAT CAC AGT CCA
CPB
NOTE: B = biotinTEG; P = triethyleneglycyl
Sequence Type: probe
SEQ ID 13
5′ ACG GTA GGT CGG TCG CAT AGC AAT AGG AGT ACC TGA GAT
GTA GCA GAA AT
Sequence Type: primer
SEQ ID 14
5′ CGG TAG GTC GGT CGC ATA GCC TGA CCT TAA GTT GTT CTT
CCA AAG CAG
Sequence Type: primer
SEQ ID 15
5′ AGG TCC ACT GCG TCC GAT GTC GTT GTT GCA TTT GTC TGT
TTC AGT TAC
Sequence Type: primer
SEQ ID 16
5′ AGG TCC ACT GCG TCC GAT GTA TCC ACT GGA GAT TTG TCT
GCT TGA G
Sequence Type: primer
SEQ ID 17
5′ B CCG GAT ACC CAG TTT CTC C
NOTE: B = biotinTEG
Sequence Type: primer
SEQ ID 18
5′ B TGG GTA CCC CAG AAA CAG TC
NOTE: B = biotinTEG
Sequence Type: primer
SEQ ID 19
5′ B TCC CCG TCC TCC TGC AT
NOTE: B = biotinTEG
Sequence Type: primer
SEQ ID 20
5′ B AGG AAG GCC TCA GTC AGG TCT
NOTE: B = biotinTEG
Sequence Type: primer
SEQ ID 21
5′ AGG TCC ACT GCG TCC GAT GT
Sequence Type: primer
SEQ ID 22
5′ CGG TAG GTC GGT CGC ATA GC
Sequence Type: primer

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A microchannel sample-handling apparatus for processing a sample, comprising
(a) a microchannel device having
(i) a substrate,
(ii) an elongate or planar multisite reaction channel formed in said substrate for receiving a bulk-phase medium containing sample components, said reaction channel having a plurality of reaction regions and region-specific reagents associated with each region, for simultaneously conducted different reactions on sample components within the reaction channel,
(iii) one or more sample-preparation stations in said substrate, upstream of said reaction channel, for carrying out one or more selected sample-preparation steps effective to convert a sample to such bulk-phase medium, and
(iv) one or more product-processing stations downstream of said reaction channel, for processing products generated in one or more of said reaction regions,
means for transferring solvent or solvent components between one of said sample-preparation stations and one or more selected reaction regions in the reaction channel, and between one or more selected reaction regions in the reaction channel and one of said product-processing stations, and
a control unit for activating said transfer means, to effect transfer, in a selected reaction region, of solvent or solvent components from or to each hold or region-specific reservoir, to or from the associated reaction region.

2. The apparatus of claim 1, which further includes a second reaction chamber for receiving a second bulk-phase medium containing sample components, said second reaction channel having a plurality of reaction regions and region-specific reagents associated with each region, for simultaneously conducting different reactions on sample components within the reaction channel.

3. The apparatus of claim 2, wherein the reaction regions in the first-mentioned reaction channel are operatively connected to associated reaction regions in the second reaction channel via gated side channels, and said control means is operative to transfer reaction components directly between associated reaction regions in the two reaction channels.

4. The apparatus of claim 2, wherein reaction regions in the first-mentioned reaction channel are operatively connected to reaction regions in the second reaction channel via a common hold reservoir which receives sample components from reaction regions in one reaction channel, and supplies the combined components to reaction regions in the other reaction channel.

5. The apparatus of claim 2, wherein the two reaction chambers are formed in different layers of the device, and reaction regions in the two channels are interconnected by side channels extending between the two layers.

6. The apparatus of claim 5, wherein at least one of the two reaction channels include capillary-tube ports adapted to receive a capillary tube therein, for supplying or removing a selected reagent or component to or from that port.

7. The apparatus of claim 1, wherein at least some of said stations and the reaction channel include capillary-tube ports adapted to receive a capillary tube therein, for supplying or removing a selected reagent or component to or from that port.

8. The apparatus of claim 1, wherein said sample-preparation stations include at least one of a cell-culture station, a station at which cells grown in the cell-culture station are lysed, and a reservoir containing lysing medium.

9. The apparatus of claim 1, wherein said product-processing stations include a station selected from the group consisting of:
(a) a waste reservoir for receiving selected components from the reaction regions;
(b) a capture station at which selected components from the reaction channel are captured and concentrated;
(c) an assay-reagent reservoir from which assay components are added to sample components;
(d) an assay station from which assay components are added to sample components;
(e) a separation channel at which sample components can be separated; and
(e) a second multisite reaction channel.

10. The apparatus of claim 9, wherein which further includes a detector for detecting the presence of absence of selected components contained in selected reaction regions, and the control unit is operable to move selected reaction components to other stations in the device, based on the presence of absence of such detected components.

11. The apparatus of claim 5, wherein the two layers are detachable reaction modules, allowing one module to be replaced by another during a sample-processing operation, where the two modules include alignable fluid-transfer channels and alignment structure for placing the modules in an aligned condition, allowing fluid transfer across the aligned channels.

12. A method of analyzing components in a sample, comprising
applying the sample to the microchannel device in the apparatus of claim 1, operating the apparatus to process the sample, forming a bulk-phase medium containing sample components,
operating the apparatus to transfer the bulk phase medium into the multisite reaction channel in the apparatus, under conditions that promote simultaneous reactions with sample components and region-specific reagents in the channel,
operating the device to transfer one or more reacted components from the reaction channel into a processing station, to achieve at least one of the following results:
(a) removal of one or more reacted components produced in the reaction channel;
(b) assay of one or more reacted components produced in the reaction channel;
(c) further reaction of one or more reacted components produced in the reaction channel,
(d) mixing of two or more selected components contained or produced in the reaction channel;
(e) transfer of one or more selected components produced in the reaction channel to selected reaction region(s) in a second multisite reaction channel.in the device;
(f) separation of components contained in the reaction channel, or produced subsequently in the device, a separation medium; and
(g) detection of components contained in the reaction channel, or produced subsequently in the device, by a detector in the device.

13. The method of claim 12, wherein said operating includes assaying one or more components produced in selected regions of the reaction channel; and based on the results of said assaying, transferring selected components to another station in the device.

14. The method of claim 12, which includes transferring reaction components from one or more regions in one reaction channel to one or more regions in a second reaction channel in the apparatus.


SEQUENCE LISTING (22 sequences)

SEQ ID NO: 1 - DNA, 40 bp, Artificial Sequence
  probe
aacagctatgaccatgcgccagggttttcccagtcacgac

SEQ ID NO: 2 - DNA, 45 bp, Artificial Sequence
  primer
cctggcgcatggtcatagcttcacccacactgtgcccatctacga

SEQ ID NO: 3 - DNA, 37 bp, Artificial Sequence
  primer
cctggcgcatggtcatagctcggaaccgctcattgcc

SEQ ID NO: 4 - DNA, 41 bp, Artificial Sequence
  probe
baacagctatgaccatgcgccagggttttcccagtcacgac

SEQ ID NO: 5 - DNA, 20 bp, Artificial Sequence
  primer
cctggcgcatggtcatagct

SEQ ID NO: 6 - DNA, 40 bp, Artificial Sequence
  probe
gtcgtgactgggaaaaccctggcgcatggtcatagctgtt

SEQ ID NO: 7 - DNA, 40 bp, Artificial Sequence
  probe
acatcggacgcagtggacctcacgtctacaagtcgcctga

SEQ ID NO: 8 - DNA, 20 bp, Artificial Sequence
  primer
aggtccactgcgtccgatgt

SEQ ID NO: 9 - DNA, 40 bp, Artificial Sequence
  probe
ctgatgccgagagctgccaagcccatatacgatgcctcga

SEQ ID NO: 10 - DNA, 45 bp, Artificial Sequence
  primer
ttggcagctctcggcatcagtcatccatcatcttcggcagattaa

SEQ ID NO: 11 - DNA, 50 bp, Artificial Sequence
  primer
ttggcagctctcggcatcagcaggcggtagagtatgccaaatgaaaatca

SEQ ID NO: 12 - DNA, 40 bp, Artificial Sequence
  probe
gctatgcgaccgacctaccgtttgagccatcacagtccac

SEQ ID NO: 13 - DNA, 50 bp, Artificial Sequence
  primer
acggtaggtcggtcgcatagcaataggagtacctgagatgtagcagaaat

SEQ ID NO: 14 - DNA, 48 bp, Artificial Sequence
  primer
cggtaggtcggtcgcatagcctgaccttaagttgttcttccaaagcag

SEQ ID NO: 15 - DNA, 48 bp, Artificial Sequence
  primer
aggtccactgcgtccgatgtcgttgttgcatttgtctgtttcagttac

SEQ ID NO: 16 - DNA, 46 bp, Artificial Sequence
  primer
aggtccactgcgtccgatgtatccactggagatttgtctgcttgag

SEQ ID NO: 17 - DNA, 19 bp, Artificial Sequence
  primer
ccggatacccagtttctcc

SEQ ID NO: 18 - DNA, 20 bp, Artificial Sequence
  primer
tgggtaccccagaaacagtc

SEQ ID NO: 19 - DNA, 17 bp, Artificial Sequence
  primer
tccccgtcctcctgcat

SEQ ID NO: 20 - DNA, 21 bp, Artificial Sequence
  primer
aggaaggcctcagtcaggtct

SEQ ID NO: 21 - DNA, 20 bp, Artificial Sequence
  primer
aggtccactgcgtccgatgt

SEQ ID NO: 22 - DNA, 20 bp, Artificial Sequence
  primer
cggtaggtcggtcgcatagc